(19) United States
(12) Patent Application Publication
(10) Pub. No.: US 2017/0225161 A1
(43) Pub. Date: Aug. 10, 2017

(54) THE PUMPING LID: DEVICES AND METHODS FOR PROGRAMMABLE GENERATION OF POSITIVE AND NEGATIVE PRESSURES

(71) Applicant: CALIFORNIA INSTITUTE OF TECHNOLOGY, Pasadena, CA (US)

(72) Inventors: Stefano BEGOLO, Inglewood, CA (US); Dmitriy V. ZHUKOV, Pasadena, CA (US); Daan WITTERS, Pasadena, CA (US); Erik B. JUE, Pasadena, CA (US); Rustem F. ISMAGILOV, Altadena, CA (US)

(21) Appl. No.: 15/504,283

(22) PCT Filed: Aug. 14, 2015
(86) PCT No.: PCT/US15/45405

§ 371 Date: Feb. 15, 2017

Related U.S. Application Data
(60) Provisional application No. 62/038,036, filed on Aug. 15, 2014.
(60) Provisional application No. 62/197,468, filed on Jul. 27, 2015.

(51) Int. Cl. B01L 3/00; C12Q 1/68

(52) U.S. Cl. CPC B01L 3/50273; B01L 3/502715; C12Q 1/686; B01L 3/502776; B01L 3/502746; B01L 2300/14; B01L 2400/0487; B01L 2300/041; B01L 2200/025; B01L 2300/046

(57) ABSTRACT

Provided herein are devices and methods for generating positive and negative pressures. The devices and methods are suited for the generation of pressures; in particular, the pressures generated can be useful for controlling the flow of fluids, such as in fluidic device.

### CROSS-REFERENCE

This application claims the benefit of U.S. Provisional Application No. 62/197,468, filed 27 Jul. 2015, and of U.S. Provisional Application No. 62/038,036, filed 15 Aug. 2014, each of which application is incorporated herein by reference.

### STATEMENT AS TO FEDERALLY SPONSORED RESEARCH

This invention was made with the support of the United States Government under award number DGE-1144469 by the National Science Foundation Graduate Research Fellowships Program, and under cooperative agreement number HR0011-11-2-0006 by the Defense Advanced Research Projects Agency. The government has certain rights in the invention.

### BACKGROUND OF THE INVENTION

Microfluidic devices can be powerful tools for the analysis of samples. Samples, such as those from subjects or environmental sources, can be analyzed for the presence of various compounds and organisms. Patients can be diagnosed for diseases, including infectious diseases and genetic diseases.

Many microfluidic devices developed in the past two decades rely on external equipment for operation. Such requirements can limit the utility of these techniques in point-of-care settings, limited resource settings, and other environments with difficult or no access to necessary resources.

### SUMMARY OF THE INVENTION

What is needed, therefore, is a technique for applying pressures and controlling fluid flow without the need for substantial external equipment such as pumps, vacuums, and other pressure sources.

An aspect of the present disclosure provides a device for altering a pressure in a chamber, the device comprising: a) a guiding structure comprising a first part and a second part, wherein the first part comprises at least one docking structure and the second part comprises a guide that engages with the at least one docking structure, wherein the guide comprises (i) two or more docking positions for the at least one docking structure, and (ii) a pathway connecting a first docking position of the two or more docking positions to a second docking position of the two or more docking positions; b) a lid comprising one of the first part or the second part of the guiding structure; and c) a vessel comprising an open cavity and one of the second part or the first part of the guiding structure, such that between the lid and the vessel, the device includes a first part and second part of the guiding structure, wherein the lid is capable of forming an airtight seal with the open cavity of the vessel, thereby defining a chamber, wherein motion of the lid relative to the vessel is guided by the pathway, and wherein, when the docking structure is in the first docking position, the chamber has a first volume and, when the docking structure is in the second docking position, the chamber has a second volume that is different from the first volume, wherein a change in volume produces a pressure change in the chamber.

An aspect of the present disclosure provides a device for altering a pressure in a chamber, the device comprising: a lid, a cover, and a vessel comprising one or more compartments, a first compartment of the one or more compartments containing a volatile material, wherein an airtight seal is formed between the lid and the vessel, thereby defining a chamber, wherein when the cover is in a first position, the cover obstructs fluid communication between the one or more compartments and the chamber, and when the cover is in a second position, the compartments are in fluid communication with each other and with the chamber and the volatile material produces a vapor pressure in the chamber.

In some embodiments of aspects provided herein, the first volume is greater than the second volume. In some embodiments of aspects provided herein, the first volume is less than the second volume. In some embodiments of aspects provided herein, the first volume in the cavity when the at least one docking structure is in the first docking position is different from a third volume in the chamber when the at least one docking structure is in the second docking position. In some embodiments of aspects provided herein, the lid further comprises a filter with a removable seal. In some embodiments of aspects provided herein, the guide comprises a third docking position and wherein the pathway connects the second docking position to the third docking position. In some embodiments of aspects provided herein, a fourth volume in the chamber when the at least one docking structure is in the third docking position is less than the second volume. In some embodiments of aspects provided herein, a fourth volume in the cavity when the at least one docking structure is in the third docking position is greater than the second volume. In some embodiments of aspects provided herein, the lid comprises the cover. In some embodiments of aspects provided herein, the device further comprises a) a guiding structure comprising a first part and a second part, wherein the first part comprises at least one docking structure and the second part comprises a guide that engages with the at least one docking structure, wherein the guide comprises (i) two or more docking positions for the at least one docking structure, and (ii) a pathway connecting a first docking position of the two or more docking positions to a second docking position of the two or more docking positions; b) a lid comprising one of the first part or second part of the guiding structure; and c) a vessel comprising an open cavity and one of the second part or first part of the guiding structure, such that between the lid and vessel, the device includes a first part and second part. In some embodiments of aspects provided herein, motion of the lid is guided by the pathway. In some embodiments of aspects provided herein, the device further comprises a port that is in fluid communication with at least one of the one or more compartments. In some embodiments of aspects provided herein, the volatile material is a halogenated hydrocarbon. In some embodiments of aspects provided herein, the volatile material is perfluorohexane. In some embodiments of aspects provided herein, the vapor pressure is at least about 1 kPa. In some embodiments of aspects provided herein, the device further comprises: an additional lid, an additional cover, and an additional vessel comprising one or more compartments, a first compartment of the one or more compartments containing an additional volatile material, wherein an airtight seal is formed between the additional lid and the additional vessel, thereby defining an additional cavity, wherein when the additional cover is in a first position, the additional cover obstructs fluid communication between the compartments and the additional cavity, and when the additional cover is in a second position, the compartments are in fluid communication with each other and with the additional cavity and the additional volatile material expands into the additional cavity and increases a pressure in the additional cavity. In some embodiments of aspects provided herein, the motion comprises rotation. In some embodiments of aspects provided herein, the lid comprises the first part of the guiding structure and the vessel comprises the second part of the guiding structure. In some embodiments of aspects provided herein, the vessel comprises the first part of the guiding structure and the lid comprises the second part of the guiding structure. In some embodiments of aspects provided herein, the at least one docking structure comprises at least one pin. In some embodiments of aspects provided herein, the first part of the guiding structure comprises at least two docking structures. In some embodiments of aspects provided herein, the guide comprises three or more docking positions. In some embodiments of aspects provided herein, the device further comprises a channel in fluid communication with the vessel. In some embodiments of aspects provided herein, the channel is less than 10 mm wide. In some embodiments of aspects provided herein, the vessel comprises a nucleic acid amplification reagent. In some embodiments of aspects provided herein, the vessel comprises a nucleic acid amplification primer specific for an infectious disease. In some embodiments of aspects provided herein, the vessel comprises a polymerase chain reaction reagent. In some embodiments of aspects provided herein, the vessel holds a sample. In some embodiments of aspects provided herein, the sample has a volume of less than about 1 mL. In some embodiments of aspects provided herein, the sample has a volume of less than about 100 μL. In some embodiments of aspects provided herein, the device further comprises a microfluidic device. In some embodiments of aspects provided herein, the device has a weight less than about 50 g.

An aspect of the present disclosure provides a device for altering a pressure in a chamber, the device comprising: a) a plurality of vessels, each comprising an open cavity; and b) a lid capable of forming an airtight seal with each open cavity of the plurality of vessels, thereby defining a plurality of chambers, wherein, when the lid is in a first position, the plurality of chambers each have a first volume and, when the lid is in a second position, the plurality of chambers each have a second volume that is different from the first volume, wherein a change in volume produces a pressure change in the plurality of chambers.

In some embodiments of aspects provided herein, a first chamber and a second chamber of the plurality of chambers are connected by the lid.

An aspect of the present disclosure provides a method of altering a pressure in a chamber, the method comprising: a) providing a device, comprising: a guiding structure comprising a first part and a second part, wherein the first part comprises at least one docking structure and the second part comprises a guide that engages with the at least one docking structure, wherein the guide comprises (i) two or more docking positions for the at least one docking structure, and (ii) a pathway connecting a first docking position of the two or more docking positions to a second docking position of the two or more docking positions; a lid comprising one of the first part or the second part of the guiding structure; and a vessel comprising an open cavity and one of the second part or the first part of the guiding structure, such that between the lid and the vessel, the device includes a first part and second part, wherein an airtight seal is formed between the lid and the vessel, thereby defining a chamber, and wherein motion of the lid is guided by the pathway; and b) moving the lid from the first docking position to the second docking position, wherein a first volume in the chamber when the at least one docking structure is in the first docking position is different from a second volume in the chamber during the moving, wherein a change in volume produces a pressure change in the chamber.

An aspect of the present disclosure provides a method of altering a pressure in a chamber, the method comprising: a) providing a device comprising: a lid, a cover, and a vessel comprising one or more compartments, a first compartment of the one or more compartments containing a volatile material, wherein an airtight seal is formed between the lid and the vessel, thereby defining a chamber; and b) moving the lid from a first docking position to a second docking position, wherein when the at least one docking structure is in the first docking position the compartments are not in fluid communication with each other or with the chamber, and when the at least one docking structure is in the second docking position the compartments are in fluid communication with each other and with the chamber and the volatile material produces a vapor pressure in the chamber.

In some embodiments of aspects provided herein, the moving compresses the chamber, and the first volume is greater than the second volume. In some embodiments of aspects provided herein, the moving decompresses the chamber, and the first volume is less than the second volume. In some embodiments of aspects provided herein, the method further comprises moving the lid from the second docking position to a third docking position. In some embodiments of aspects provided herein, the moving the lid from the second docking position to a third docking position compresses the chamber to a third volume that is less than the second volume. In some embodiments of aspects provided herein, the moving the lid from the second docking position to a third docking position decompresses the chamber to a third volume that is greater than the second volume. In some embodiments of aspects provided herein, the device further comprises a guiding structure comprising a first part and a second part, wherein the first part comprises at least one docking structure and the second part comprises a guide that engages with the at least one docking structure, wherein the guide comprises (i) two or more docking positions for the at least one docking structure, and (ii) a pathway connecting a first docking position of the two or more docking positions to a second docking position of the two or more docking positions. In some embodiments of aspects provided herein, motion of the lid is guided by the pathway. In some embodiments of aspects provided herein, the volatile material is a halogenated hydrocarbon. In some embodiments of aspects provided herein, the volatile material is perfluorohexane. In some embodiments of aspects provided herein, the vapor pressure is at least about 1 kPa. In some embodiments of aspects provided herein, the lid comprises the first part of the guiding structure and the vessel comprises the second part of the guiding structure. In some embodiments of aspects provided herein, the vessel comprises the first part of the guiding structure and the lid comprises the second part of the guiding structure. In some embodiments of aspects provided herein, the at least one docking structure comprises at least one pin. In some embodiments of aspects provided herein, the first part of the guiding structure comprises at least two docking structures. In some embodiments of aspects provided herein, the guide comprises three or more docking positions. In some embodiments of aspects provided herein, the lid further comprises a filter with a removable seal. In some embodiments of aspects provided herein, the device further comprises a channel in fluid communication with the vessel. In some embodiments of aspects provided herein, the channel is less than 10 mm wide. In some embodiments of aspects provided herein, the vessel comprises a nucleic acid amplification reagent. In some embodiments of aspects provided herein, the vessel comprises a nucleic acid amplification primer specific for an infectious disease. In some embodiments of aspects provided herein, the vessel comprises a polymerase chain reaction reagent. In some embodiments of aspects provided herein, the vessel holds a sample. In some embodiments of aspects provided herein, the sample has a volume of less than about 1 mL. In some embodiments of aspects provided herein, the sample has a volume of less than about 100 μL. In some embodiments of aspects provided herein, the device further comprises a microfluidic device. In some embodiments of aspects provided herein, the device has a weight less than about 50 g. In some embodiments of aspects provided herein, the moving comprises rotating. In some embodiments of aspects provided herein, the method further comprises a channel in fluid communication with the vessel.

An aspect of the present disclosure provides a method of altering a pressure in a chamber, the method comprising: a) providing a device, comprising: a plurality of vessels, each comprising an open cavity; and a lid, wherein an airtight seal is formed between the lid and the plurality of vessels, thereby defining a plurality of chambers; and b) moving the lid from a first position to a second position, wherein a first volume in each of the plurality of chambers when the lid is in the first position is different from a second volume in each of the plurality of chambers during the moving, wherein a change in volume produces a pressure change in each of the plurality of chambers.

In some embodiments of aspects provided herein, a first chamber and a second chamber of the plurality of chambers are connected by the lid.

An aspect of the present disclosure provides a device for altering pressure in a chamber, the device comprising: (a) a guiding structure comprising a first part and a second part, wherein the first part comprises at least one docking structure and the second part comprises a guide that engages with the at least one docking structure, wherein the guide comprises a pathway defining a program of axial movement; (b) a vessel comprising an open cavity and one of the first part or second part of the guiding structure; (c) a lid configured to rotate relative to the vessel, capable of forming an airtight seal with the open cavity of the vessel thereby defining a chamber, and comprising one of the second part or first part of the guidance structure, wherein if the lid comprises the first part of the guidance structure, then the vessel comprises the second part and if the lid comprises the second part of the guidance structure, then the vessel comprises the first part, wherein rotation of the lid relative to the vessel engages the docking structure with the pathway, guides the lid inward and/or outward relative to the vessel, thereby decreasing or increasing the volume of the chamber and thus altering pressure in the chamber.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The present application incorporates the following applications by reference in their entireties for any and all purposes: U.S. Application 61/516,628, “Digital Isothermal Quantification of Nucleic Acids Via Simultaneous Chemical Initiation of Recombinase Polymerase Amplification (RPA) Reactions on Slip Chip,” filed on Apr. 5, 2011; U.S. Application 61/518,601, “Quantification of Nucleic Acids With Large Dynamic Range Using Multivolume Digital Reverse Transcription PCR (RT-PCR) On A Rotational Slip Chip Tested With Viral Load,” filed on May 9, 2011; U.S. application Ser. No. 13/257,811, “Slip Chip Device and Methods,” filed on Sep. 20, 2011; international application PCT/US2010/028361, “Slip Chip Device and Methods,” filed on Mar. 23, 2010; U.S. Application 61/262,375, “Slip Chip Device and Methods,” filed on Nov. 18, 2009; U.S. Application 61/162,922, “Slip Chip Device and Methods,” filed on Mar. 24, 2009; U.S. Application 61/340,872, “Slip Chip Device and Methods,” filed on Mar. 22, 2010; U.S. application Ser. No. 13/440,371, “Analysis Devices, Kits, And Related Methods For Digital Quantification Of Nucleic Acids And Other Analytes,” filed on Apr. 5, 2012; and U.S. application Ser. No. 13/467,482, “Multivolume Devices, Kits, Related Methods for Quantification and Detection of Nucleic Acids and Other Analytes,” filed on May 9, 2012; U.S. application Ser. No. 13/868,028, “Fluidic Devices and Systems for Sample Preparation or Autonomous Analysis,” filed on Apr. 22, 2013; U.S. application Ser. No. 13/868,009, “Fluidic Devices for Biospecimen Preservation,” filed on Apr. 22, 2013; international application PCT/US2013/063594, “Methods and Systems for Microfluidics Imaging and Analysis,” filed on Oct. 4, 2013; international application PCT/US2014/034728, “Parallelized Sample Handling,” filed on Apr. 18, 2014; international application PCT/US2014/047092, “Digital Assay for Quantifying and Concentrating Analytes,” filed on Jul. 17, 2014; international application PCT/US2014/056401, “System and Method for Movement and Timing Control,” filed on Sep. 18, 2014; U.S. Application 62/096,131, “Devices and Methods for Autonomous Measurements,” filed on Dec. 23, 2014; and U.S. Application 62/135,041, “Devices and Methods for Autonomous Measurements,” filed on Mar. 18, 2015.

### BRIEF DESCRIPTION OF THE DRAWINGS

Novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings or figures (also referred to herein as “FIG.” and “FIGS.”), of which:

FIG. 1A illustrates an exemplary device that can be used to generate positive pressure in a vessel. FIG. 1B illustrates a device that can be used to generate negative pressure in a vessel. FIG. 1C shows exemplary results for generation of positive and negative pressure with different combinations of pumping lids and vessels tested.

FIG. 2A-FIG. 2H illustrate design and operation of exemplary devices for producing multiple pressure values in a single device using a lid and vessel.

FIG. 3A and FIG. 3B show exemplary experimental and quantitative data for a model describing pumping with a pumping lid as a function of hydraulic resistance of the channel and properties of the fluid.

FIG. 4A shows an exemplary schematic of a pumping approach using multiple solutions in the same device. FIG. 4B shows experimental photographs illustrating production of nanoliter plugs. FIG. 4C illustrates a parallel laminar flow profile of three separate streams of aqueous solutions.

FIG. 5A illustrates exemplary schematics of the setup used for formation of different flow profiles in a single device using composite pumping lids. FIG. 5B illustrates an exemplary junction at which parallel laminar flow is produced. FIG. 5C shows exemplary flow profiles formed with different composite lids.

FIG. 6A illustrates an example of fluidic device sample loading via negative pressure. FIG. 6B shows a photograph of a multivolume SlipChip device loaded with a negative pressure pumping lid method.

FIG. 7A and FIG. 7B illustrate an exemplary device that can be used to generate pressure using vapor liquid equilibrium. FIG. 7C illustrates an exemplary experimental pressure profile obtained by performing the steps shown in FIG. 7B. FIG. 7D shows an exemplary pressure profile obtained when pumping a 2 mL sample volume through a microfluidic device.

FIG. 8 provides an exemplary schematic representation of parameters that can be used for calculating positive pressure generation.

FIG. 9 provides an exemplary schematic representation of the parameters that can be used for the calculation of negative pressure generation.

FIG. 10 illustrates an exemplary experimental setup for flow rate measurement.

FIG. 11 illustrates an exemplary device that can be used for the generation of positive pressure using magnetic force.

FIG. 12 illustrates devices that can be used for the generation of positive and negative pressure of a vessel using springs.

FIG. 13A provides an example of elastic deformation of a spherical vessel for pressure stabilization during pumping. FIG. 13B shows exemplary graphs of pressure inside a spherical elastic element as a function of its equibiaxial deformation.

FIG. 14 provides an exemplary device, comprising a pumping lid and a filter to prevent contamination.

FIG. 15A provides an exemplary schematic outline of the loading of a SlipChip device with a pumping lid. FIG. 15B provides a photograph of the exemplary loading of a SlipChip device with a pumping lid. FIG. 15C provides an exemplary schematic of pipette tip loading in conjunction with a pumping lid.

FIG. 16A illustrates an exemplary pumping lid placed over the arm of the C-clamp rather than directly over the inlet. FIG. 16B illustrates an exemplary pumping lid with a lock.

FIG. 16C illustrates an exemplary 2-piece pumping lid with a filter that can be used for thermocycling.

FIG. 17A illustrates an exemplary pumping lid with a sloping profile. FIG. 17A illustrates an exemplary pumping lid with a sloping profile that can be used in combination with a clamp. FIG. 17C illustrates an exemplary pumping lid with a sloping profile that is equipped with a filter to prevent contamination. FIG. 17D illustrates an exemplary pumping lid with a sloping profile that is integrated with a tightening mechanism and equipped with a filter.

FIG. 18 shows a computer system that is programmed or otherwise configured to operate a device or analyze results from a device of the present disclosure.

### DETAILED DESCRIPTION OF THE INVENTION

Many microfluidic devices developed in the past two decades rely on external equipment for operation. However, many applications can benefit from equipment-free pumping, such as in limited resource settings. Thus, it is desirable to develop an equipment-free pumping device and method. Disclosed herein are devices and systems for equipment-free generation of positive and negative pressures in a microfluidic device using a pumping lid. These pumping methods can be used in a variety of microfluidic applications, such as including the production of droplets, control of laminar flow profiles, and loading of microfluidic (e.g., SlipChip) devices. These devices can be made using portable, lightweight, and disposable parts that can be integrated with existing microfluidic devices to simplify workflow and eliminate the need for pumping equipment.

One type of pumping lid described herein can be used to produce predictable positive or negative pressures via controlled compression or expansion of gases. Pressures can be pre-programmed by the geometry of the parts. A guiding structure can engage with docking structures of a lid, wherein the relative movement of the lid can affect the pressure within a vessel. Depending on the geometry of the pathway connecting the docking positions, the pressure within the vessel can incrementally increase, incrementally decrease, or reversibly increase and decrease from one docking position to the next docking position. Using multiple lids or a composite lid with different ports (e.g., inlets, outlets) can enable several solutions to be pumped independently in a single device.

A second type of pumping lid can employ volatile materials that off-gas at significant enough rates to generate pressure, including pressure sufficient to move samples through a microfluidic device. Initially, a volatile material can be stored in sealed compartments separated from a sample. A cover can be actuated to effect fluid communication between compartments. For example, a cover can obstruct gaseous communication between the compartment and the vessel (e.g., in a first position). The cover or seal can be pierced or removed to effect gaseous communication between the compartment and the vessel (e.g., in a second position). The vapor pressure generated by a volatile material can then be used to pump a sample. Volatile materials include those with a tendency to vaporize and produce a vapor pressure. Volatile materials can include liquids as well as solids (e.g., dry ice, ammonium chloride). Volatile materials can transition from liquid to vapor, or can sublimate from solid to vapor.

### Definitions

As used herein, “about” means +/−10% of the recited value.

As used herein, “or” includes “and/or.”

By “between” is meant a relative position in which an intermediate structure separates a first and a second structure. For instance, in a device including an intermediate substrate disposed between a first and a second substrate, the term “between” provides the relative positional relationship of the first, second, and intermediate substrates and in no way signifies that the first substrate must necessarily be the top or uppermost substrate in the device.

By “engage” is meant a physical interaction between two components or structures. This physical interaction can be direct (e.g., where a first component interacts with a second component) or indirect (e.g., where a first component interacts with an interleaving component, which in turn interacts with a second component).

### Devices

### Pumping Lids and Vessels

Described herein are devices and systems for generating pressure, such as by controlled compression or expansion of gas. The pressure can serve as a motive force for transferring solutions or other fluids to and from different locations in a device or system. The pumping lid approach can be used to pump fluids at a predictable flow rate. Flow can be generated in any channel or compartment, including, for example, tubing, microfluidic channels, chambers, microfluidic chambers, or containers.

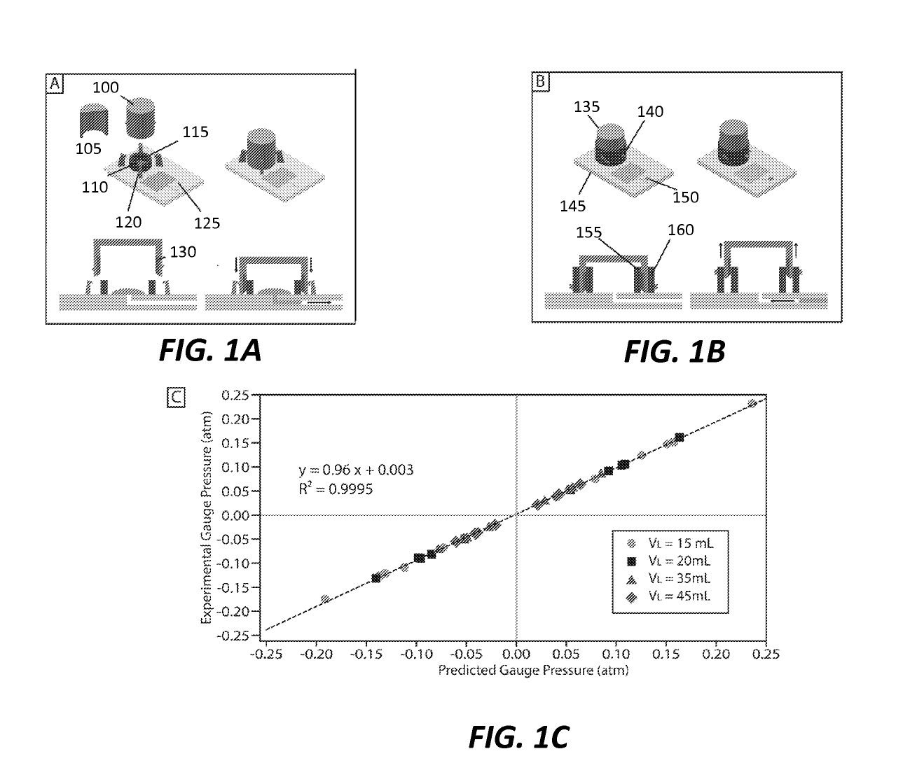

Such devices can comprise a lid (e.g., a pumping lid) and a vessel. A pumping lid can be used to control compression or expansion of gas (see, e.g., FIG. 1A-FIG. 1C), thereby controlling a pressure within a vessel. A pumping lid 100 can comprise an empty cavity 105, with a volume of this cavity defined as V_{L}. A vessel can comprise a part having a cavity 115, with a volume of the cavity is defined as V_{C}, while the volume of the vessel material (e.g., its walls) is defined as V_{W}.

To generate positive pressure, a sample can be placed at a port, such as a device inlet 110, and the pumping lid 100 can be placed on the vessel 115 (see, e.g., FIG. 1A). When the lid is pushed down, the air in the lid's cavity can be isolated and compressed, creating positive-gauge pressure. The lid's position can held by friction, but to increase robustness, guiding and docking structures 120, 130 (such as rails, pins, deformable parts that create docking mechanisms) can be integrated into the design of the lid or the vessel (see, e.g., FIG. 1A and FIG. 1B). To create negative pressure, a pumping lid 135 can be pre-placed on the vessel (see, e.g., FIG. 1B) and then pulled up to expand the air in the cavity. The motion of the lid can be guided by guiding structures and docking structures 140, 155, 160. Generation of positive or negative pressure can be used to control pressures within a device 145. Fluid flow can be controlled, such as through device ports 125, 150.

In another implementation, the pumping lid can comprise a plug element that fits within the interior of the vessel cavity, wherein the plug element forms an air tight seal with the interior walls of the vessel. To generate positive pressure, for example, a sample can be placed at a port and the plug element of the lid can be placed at the top opening of the vessel. When the lid is pushed down, the air in the vessel is compressed, creating positive pressure. To create negative pressure, for example, a lid with a plug element can be pre-placed deep within the vessel and then pulled up, but not out, to expand the air in the cavity.

Various types of actuation or movement of any component of the device may occur. Various types of actuation may include, but are not limited to, rotating, lifting, rolling, pushing, pulling, and ejecting. In some examples, the lid may be rotated relative to the pathway of the guiding structure. The pathway can link two or more docking positions. The lid may also move vertically, relative to the guiding structure. The guiding structure can comprise a program of vertical positions for the lid, such that rotation of the lid relative to the guiding structure will cause the lid to rise and fall, thus incrementally increasing and decreasing the volume with the air-tight vessel.

When designing a pumping lid and vessel, without being bound by theory, theoretical models such as those discussed herein can be used to predict the pressure generated by a particular lid/vessel combination, or to design a lid and vessel to achieve a particular pressure. All parameters can be tuned, and the resulting pressure for each combination can be predicted using the equations described herein.

To improve sealing between the pumping lid and the vessel, at least one of the lid or vessel can contain a deformable (e.g., soft) portion. A small overlap between the parts can be produced, so the soft portion is forced to deform when the lid is placed on the vessel, thus creating a seal (e.g., hermetic or airtight). Overlaps can be on the order of 100 μm to 200 μm. Overlaps can be on the order of about 1%-2% of the diameter of the vessels. Compression can deform the soft portion of the lid, and the material can be squeezed laterally. If this deformable material goes between the pumping lid and the base of the vessel, the material may obstruct the lid from being pushed to its final position, which can result in the obtained pressure being lower than the one predicted by a model. This effect can be minimized by ensuring that the thickness of the soft layer is significantly larger than the overlap between the lid and vessel, for example on the order of 1 mm to 1.5 mm. Another solution is to use soft layers with a tapered profile (see, e.g., FIG. 1A).

The devices described herein can comprise a vessel. The vessels can be used to hold fluids (e.g., samples or reagents). In some embodiments, a device can comprise one or more vessels. The device can include 1, 2, 3, 4, 5, 6, 7, 8, 9, 10, or more vessels. The device can include 1, 2, 3, 4, 5, 6, 7, 8, 9, 10, or more lids. Each lid can be separate from other lids.

### Docking and Guiding Structures

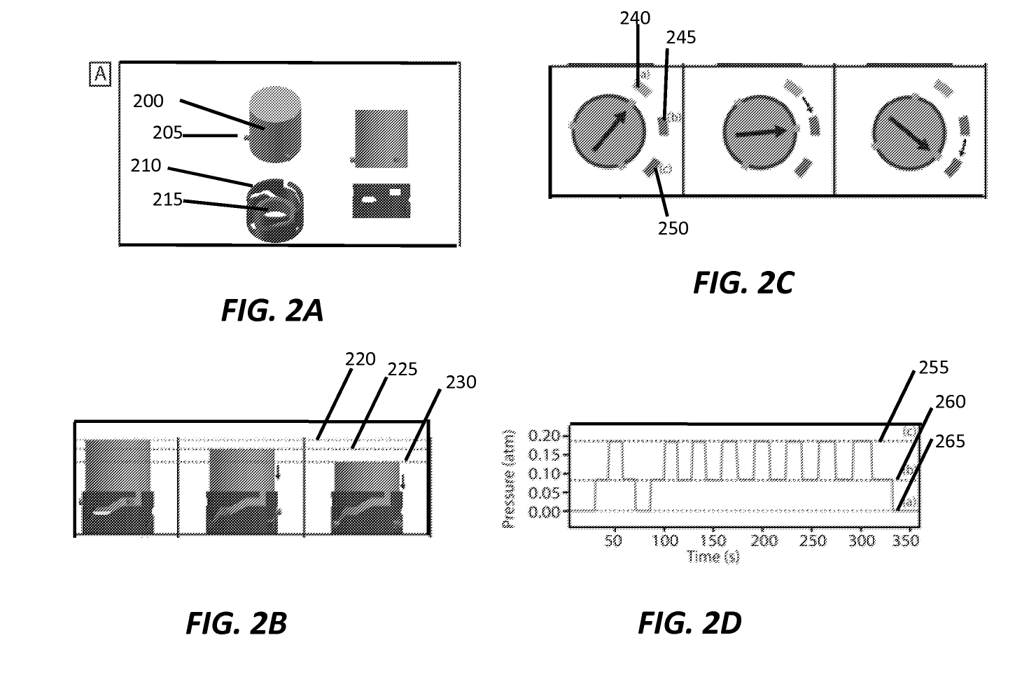

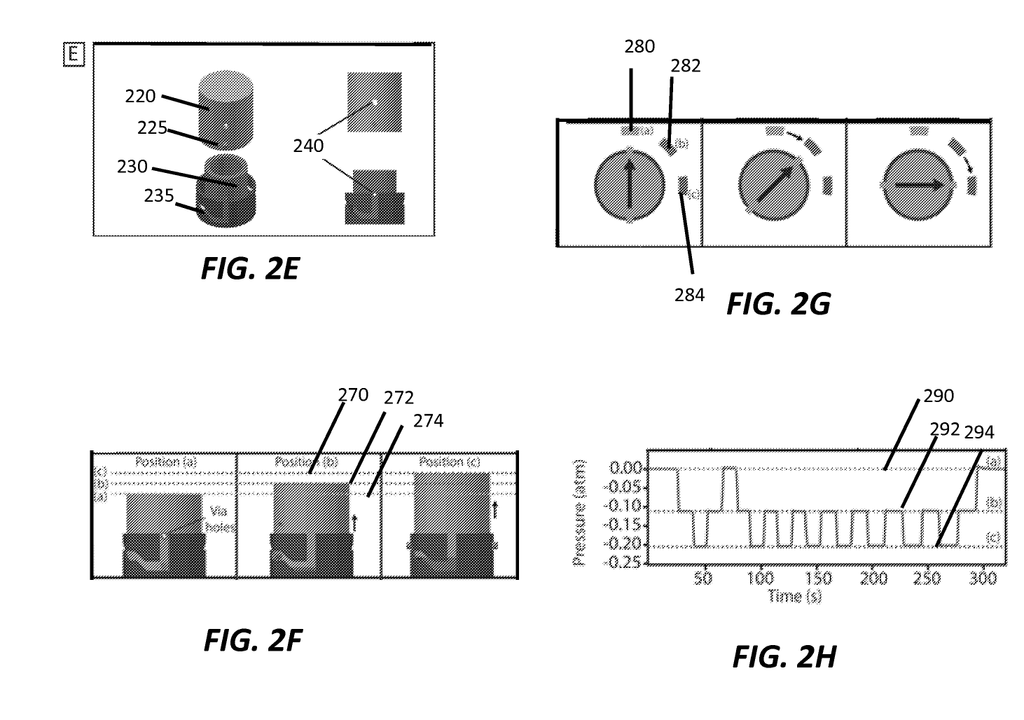

The actuation of a pumping lid can be guided by one or more guiding structures. Guiding structures can comprise guides which interface with one or more docking structures. For example, a pumping lid 200, 220 can comprise one or more docking structures (e.g., pins) 205, 225 that engage with a guide (e.g., a track or rail) 210, 230 associated with a vessel (see, e.g., FIG. 2A-FIG. 2H). Alternatively, a pumping lid can comprise a guide that engages with one or more docking structures associated with a vessel. The interaction between the docking structure(s) and the guide(s) can direct the motion of the lid (e.g., up or down) with respect to the vessel 215, 235, thereby controlling the degree of contraction or expansion within a cavity or chamber and the resulting pressure change. For example, a lid can be rotated such that the guiding structure directs the lid down (see, e.g., FIG. 2A-FIG. 2D) or up (see, e.g., FIG. 2E-FIG. 2H) as the lid rotates, thereby contracting or expanding the cavity, respectively, and generating positive or negative pressures within the cavity, respectively. A device can comprise 1, 2, 3, 4, 5, 6, 7, 8, 9, 10, or more docking structures. Docking structures can include but are not limited to pins, pegs, posts, nails, hooks, and locks.

The motion of the lid can be controlled by guides. A guide can guide the motion of a lid such that when the lid is moved in one direction (e.g., rotationally), the guide also directs the lid in another direction (e.g., up or down). Such guidance can result in the contraction or expansion of a chamber formed with the lid, thereby increasing or decreasing the pressure within the cavity. A device can comprise 1, 2, 3, 4, 5, 6, 7, 8, 9, 10, or more guides. Guides can include but are not limited to rails, tracks, slots, and grooves. A vessel can be located within a guiding structure. The guiding structure may have a nozzle for pressure measurement.

The docking structures can engage with the guide, and can rest or remain in one or more docking positions for a period of time. A guide can comprise 1, 2, 3, 4, 5, 6, 7, 8, 9, 10, or more docking positions. The pressure within a cavity or chamber can be influenced by the docking position. Docking positions can be designed to provide set points to achieve particular pressures within the cavity or chamber. Actuation of a lid such that the docking structures move through one or more docking positions with respect to the guide can be used provide a defined series of pressures within a chamber. Such a series of pressures can be used for performing operations on a device, such as the actuation of fluids within a device.

In some cases, the relative pressure of the chamber from one docking position to a subsequent docking position may always increase. In other cases, the relative pressure of the chamber from one docking position to a subsequent docking position may always decrease. In some cases, the relative pressure of the chamber from one docking position to a subsequent docking position may increase, then decrease, then increase again; in other cases, the relative pressure of the chamber from one docking position to a subsequent docking position may decrease, then increase, then decrease again. The sequence of relative pressures achieved by moving or actuating the lid and docking structures through a series of docking positions can include any series of pressure increases or decreases, in any order.

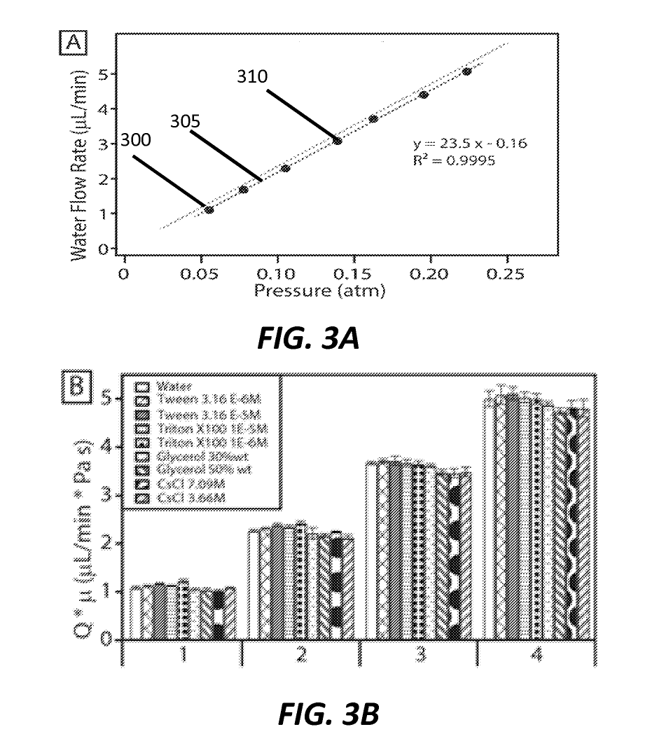

Pumping lids can be designed to be interchangeable, so the same fluidic device, with a vessel having set dimensions, can be used with different lids to generate different flow rates. Pressures can be tuned by choosing the pumping lid with the appropriate dimensions and/or by modifying the lid geometry. FIG. 3A shows a range of pressures and flow rates generated by different lid geometries in combination with the same device. Flow rates can be tuned precisely, with values ranging from a few nanoliters to more than a microliter per second, and remain consistent for long periods (e.g., hours in some cases). The same device setup can pump liquids of different density and/or surface energy with no difference in the resulting flow rate. FIG. 3B shows a comparison of flow rates for a range of different fluids with four different pumping lid geometries.

The sample volume pumped can be larger than the internal volume of the device, making the method appropriate for handling samples of various volumes, including volumes that range from a few microliters to milliliters. Both positive and negative pressures can be produced in predictable way and used to generate and control flow. While pumping is in progress, the lid can keep the sample isolated from the external environment, preventing contamination and evaporation.

### Composite Lids

Multiple lids can be used independently or can be connected in a composite lid, where the composite lid can include a single or multiple cavities and be used to simultaneously engage with multiple vessels, thereby forming multiple chambers.

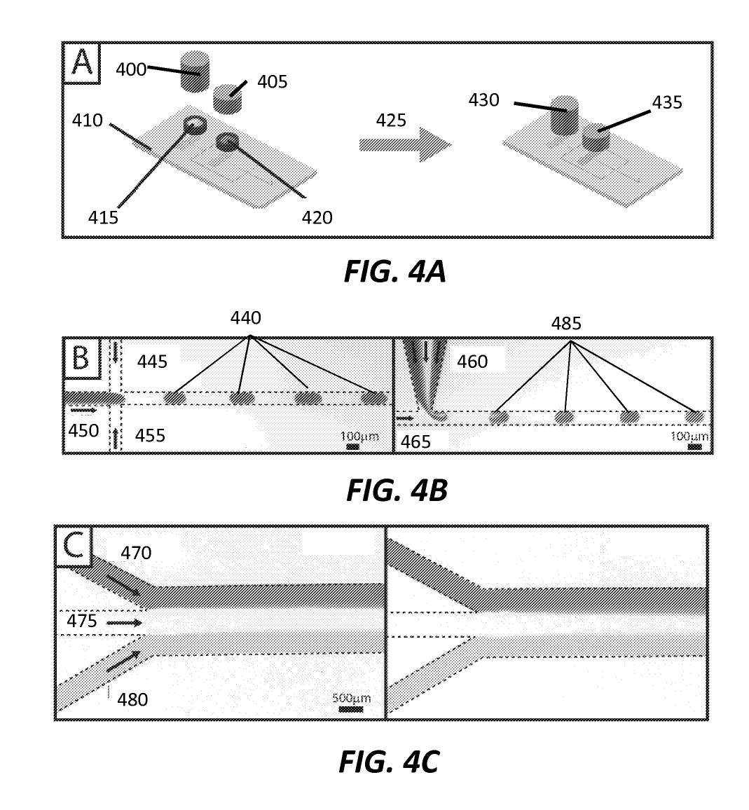

FIG. 4A-FIG. 4C illustrate an exemplary pumping lid approach to control pumping of each of several fluids with different properties in a microfluidic device. FIG. 4A shows a schematic of the pumping approach using multiple solutions in the same device. Each sample was pumped in the device with a different pumping lid, each lid producing a different pressure. On a device 410, lids 400 and 405 can be placed on top of vessels containing samples 415 and 420, respectively, to form two separate airtight chambers 430 and 435 in step 425. Each of the chambers 430 and 435 can be connected to a separate microfluidic channel to receive the pumped samples 415 and 420. FIG. 4B shows experimental photographs illustrating production of nanoliter plugs 440 from a plug fluid stream 450 driven by one pumping lid and immiscible carrier fluid streams 445 and 455 driven by another pumping lid. The right panel of FIG. 4B illustrates production of multicomponent aqueous droplets 485 in fluorinated oil using a T-junction. In this example, the plug fluid stream 460 and the immiscible carrier fluid stream 465 were pumped independently and to produce nanoliter plugs. FIG. 4C shows experimental photographs illustrating pumping lid-generated stable parallel laminar flow profile of three separate streams 470, 475, and 480 of aqueous solution after 165 minutes (2.75 hours).

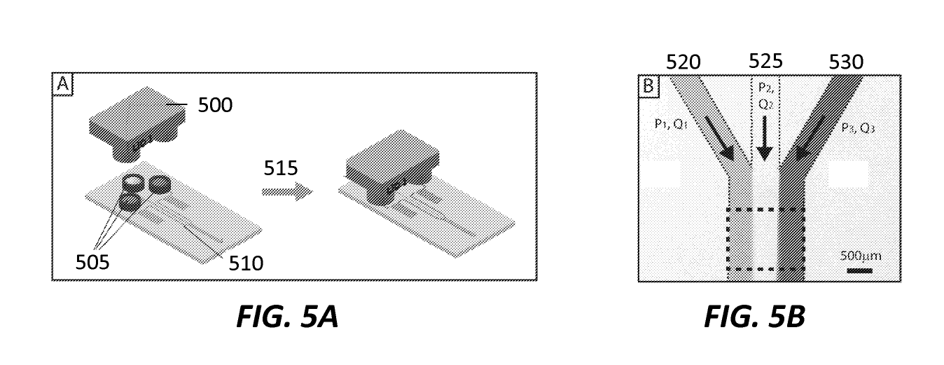

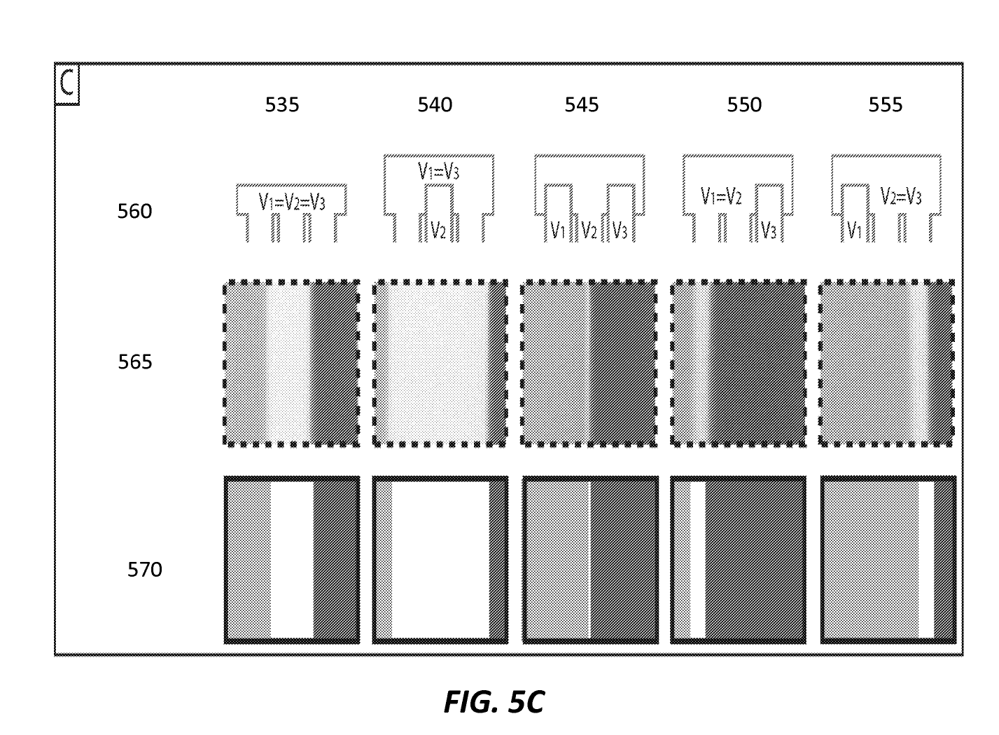

FIG. 5A-FIG. 5C illustrate exemplary production of different flow profiles in the same device using composite pumping lids. FIG. 5A illustrates schematics of the setup used for the experiments. In this experiment the microfluidic device has three vessels, each dedicated to a different aqueous solution with samples 505, but the number of vessels can be different than three. A composite lid controls the pressure at each of the three vessels, thus controlling the flow rate of each solution at each of the three inlets to the device. FIG. 5B illustrates a junction at which the three inlet branches 520, 525, and 530 combine into a single channel and the streams from the three inlets produce parallel laminar flow.

Connecting different chambers, such as via a composite lid, can be used to control the relative pressurization in the different chambers. FIG. 5C gives flow profiles produced with different composite lids 535, 540, 545, 550, and 555. The top row 560 shows the cross-section of the five different lids. The middle row 565 shows the experimental flow profiles obtained with these five lids in the same microfluidic device. The bottom row 570 shows the expected flow profiles based on the pressures produced by the lids and the device geometry.

A “composite lid,” a pumping lid with multiple sealing portions, can be used to simultaneously pressurize multiple vessels (see, e.g., FIG. 5A-FIG. 5C). The seals in the composite lid can be isolated or connected to one another. For example, if multiple inlets require identical pressures, their corresponding cavities can be linked (see, e.g., FIG. 5C). A composite lid design can be employed with at least 1, 2, 3, 4, 5, 6, 7, 8, 9, 10, or more different vessels and/or cavities to generate at least 1, 2, 3, 4, 5, 6, 7, 8, 9, 10, or more different pressures. Each composite lid can have a different geometry (see, e.g., FIG. 5C) and generate a different set of pressures at the different device inlets. These pressures can be calculated or measured, and used to predict the flow profile in the microfluidic device. Composite lids can be used, for example, to produce parallel laminar flow profiles in a microfluidic device (see, e.g., FIG. 5B).

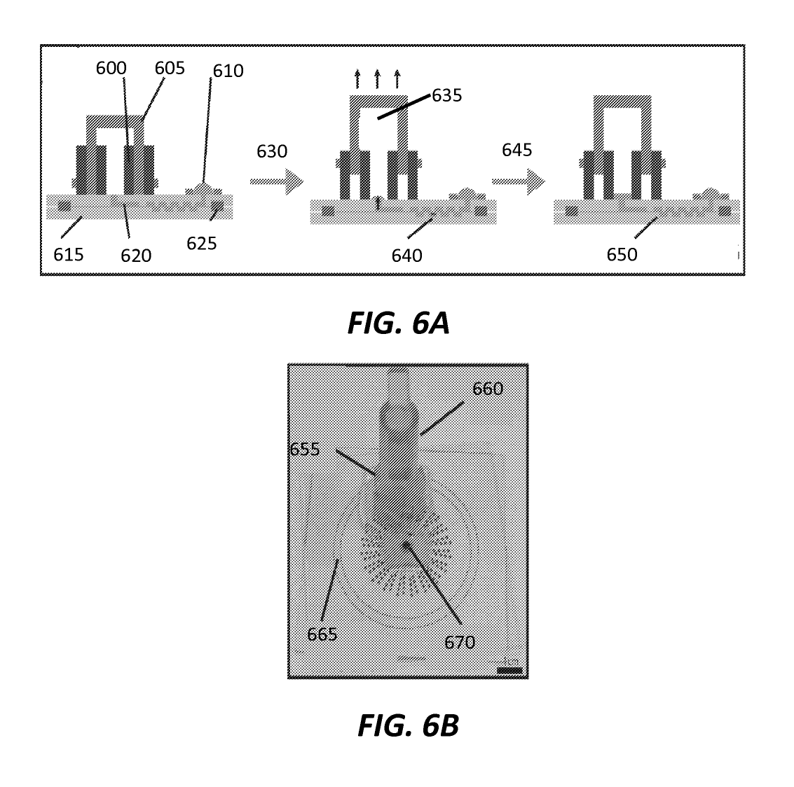

FIG. 6A illustrates an example of microfluidic device (e.g., SlipChip) sample loading by negative pressure. The lid 605 can be pre-placed on the vessel 600, and the sample 610 can be placed at a separate inlet in the device 615 (see, e.g., FIG. 6A). Oil 620 and a sealing structure 625 may be present. Pulling the lid in step 630 can create a negative gauge pressure and initiate loading. A vacuum 635 can be generated, and the sample can be loaded in channel 640. Dead-end filling 650 can ensure that the loading stops automatically in step 645 once the device is filled. FIG. 6B shows a photograph of a multivolume SlipChip microfluidic device for digital nucleic acid quantification loaded with negative pressure pumping lid method.

In one example, a device having three inlets, three vessels, and composite lids was used to produce parallel laminar flow. In general, devices with other numbers of inlets and vessels can be used. The width of each solution stream in the three-stream aqueous laminar flow was measured. The Reynolds number was always less than 1, indicating a laminar flow regime. In this example, the gauge pressures at the three inlets are defined as P₁, P₂, and P₃, while the pressure at the device outlet is zero. Fluidic resistances for the three inlet branches (before the junction) are defined as R, while the resistance of the main channel (formed by the junction of the three inlet branches) is defined as r. The fluidic resistance R of the inlet branches was intentionally set larger than the outlet resistance r, to increase the range of pressures that could be applied to the three inlets without generating back-flow in the branch with the lowest pressure.

Without being bound by theory, the prediction that for a given channel geometry, the pumping lid method would provide consistent flow rate that depends on viscosity, but not on surface energy or density of the fluid being pumped was tested. Eq. 3 was used to predict the pressure applied by the pumping lid, and Eq. 1 was used to predict hydraulic resistance R_{H} that depends on the viscosity μ and the dimensions of the channel:

$${R}_{H} = \frac{12 μ L}{{h}^{3} w ( 1 - 0.63 ( \frac{h}{w} ) )} \qquad ( Eq . 1 )$$

L defines the channel length, h the channel height, and w the width of the channel, where width is the major axis and height is the minor axis (i.e., w≧h). The volumetric flow rate can thus be predicted with Eq. 2:

$$Q = \frac{P}{{R}_{H}} = \frac{{Ph}^{3} w ( 1 - 0.63 ( \frac{h}{w} ) )}{12 μ L} \qquad ( Eq . 2 )$$

Under these conditions, theory predicts that Qᵢ is proportional to Pᵢ and can be approximated by Eq. 2. Ignoring the effects of three-dimensional diffusion and ignoring the effect of the parabolic flow profile for these wide channels, the flow profiles were predicted as described, and found to be in good agreement with experiments (see, e.g., FIG. 5C).

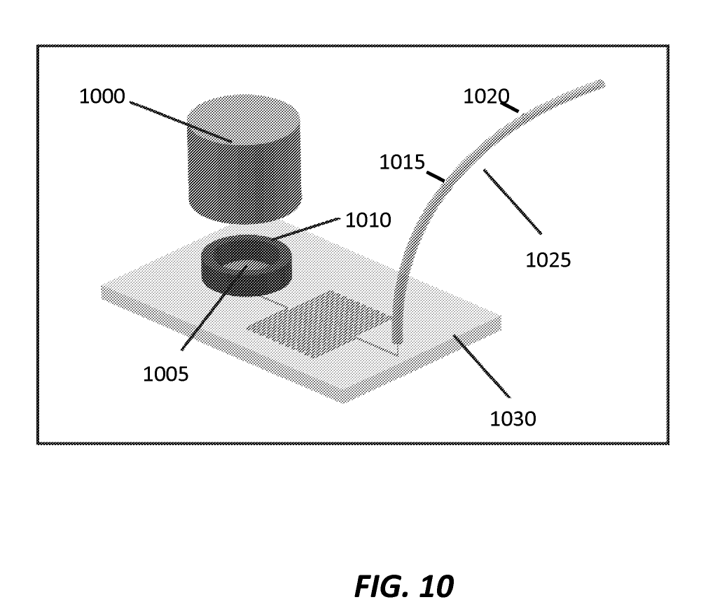

FIG. 10 provides a schematic representation of an experimental setup used for a flow rate measurement. A pumping lid 1000, vessel wall 1010, sample 1005, and tubing 1025 can be placed on a device 1030. The pumping lid can be placed onto the vessel, resulting in compression of the sample. The sample can then travel from the vessel into the tubing 1025. The time it took the air-liquid interface to travel from point 1015 to point 1020 can be recorded.

### Volatile Material Expansion and Vapor Equilibrium

The vapor pressure of a volatile material can aid the pumping process. By taking advantage of vapor-liquid equilibrium (VLE), or in certain implementations, vapor equilibrium with a solid, it is possible to pump large volumes of liquid over extended periods of time at a relatively constant pressure without the need to compress a large volume of a gas inside the device. A single lid design can be used to generate different pressures by using liquids of different vapor pressure. Additionally, a single combination of a lid design and a volatile material can be used to generate different pressures by tuning the temperature.

A volatile material is a material that can vaporize, evaporate, or sublimate relatively easily. The boiling point of a liquid can be used to measure volatility of a liquid. Some examples of volatile substances include, but are not limited to, perfluorohexane, 1,1,1,2,-tetrafluoroethane, 1,1,1,2,-tetrafluoroethane, propane, n-butane, isobutane, dimethyl ether, ethyl methyl ether, nitrous oxide, carbon dioxide, water, methanol, ethanol, n-propanol, n-butanol, chloroform, and acetone. Table 1 shows exemplary volatile materials and corresponding vapor pressures at 25° C.

TABLE 1
Examples of volatile materials and corresponding vapor pressures.

| | Vapor |
|---|---|
| | pressure |
| Material | (kPa) at 25° C. |
| perfluorohexane | 27 |
| HFA 134a (1,1,1,2,- | 666.1 |
| tetrafluoroethane) | |
| HFA | 460.06 |
| 227 (1,1,1,2,3,3,3- | |
| heptafluoropropane) | |
| propane | 945.67 |
| n-butane | 242.82 |
| isobutane | 348.03 |
| dimethyl ether | 619.84 |
| ethyl methyl ether | 195.34 |
| nitrous oxide | 5306.9 |
| carbon dioxide | 25858 |
| water | 3.17 |
| methanol | 16.92 |
| ethanol | 7.88 |
| n-propanol | 2.73 |
| n-butanol | 0.82 |
| chloroform | 25.95 |
| acetone | 30.65 |

A volatile material can produce a vapor pressure of at least about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, or 30000 kPa. A volatile material can produce a vapor pressure of at most about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, or 30000 kPa. A volatile material can produce a vapor pressure of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, or 30000 kPa. The selection of a volatile material can be dependent on the intended use of the device. For example, for devices in which samples are to be pumped quickly from a chamber to another compartment of a microdevice can have a higher vapor pressure. Alternatively, in certain implementations, a slow relatively constant flow of solution can be desirable, for example replenishing growth media for microbial incubation.

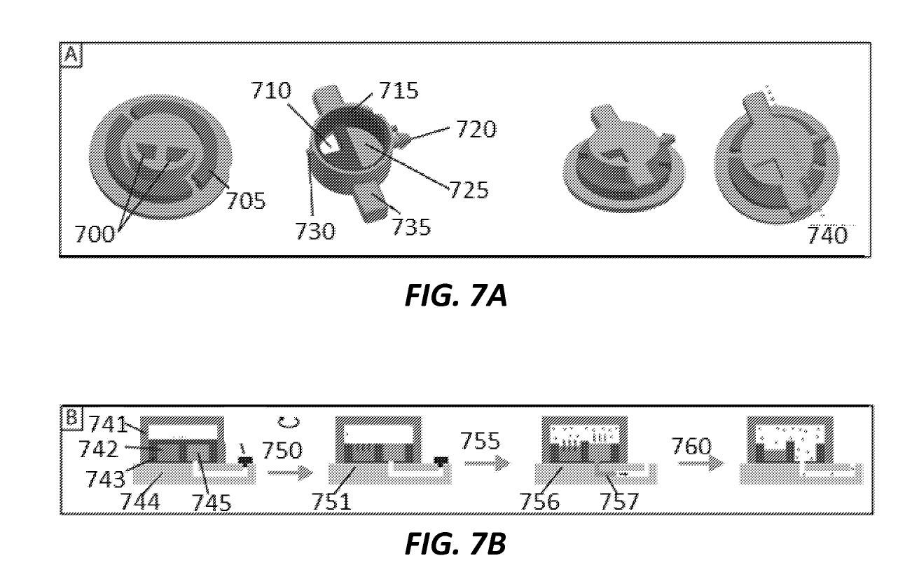

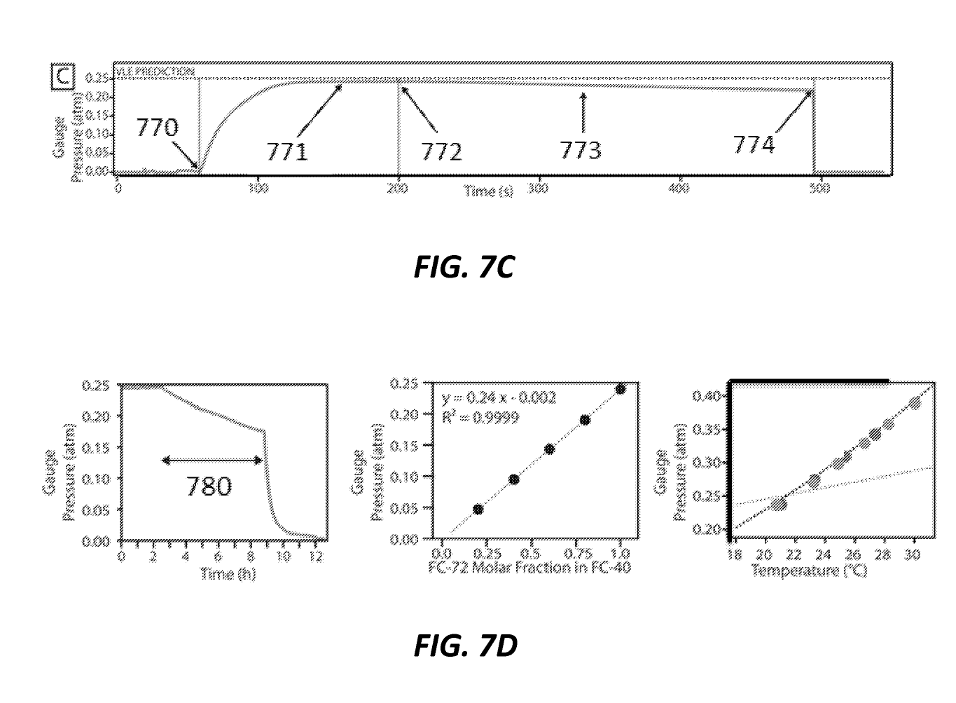

A volatile material can be separated or sealed off from the vessel chamber and connected or unsealed when pressure generation is desired. Confinement of a volatile material can be achieved by the use of any sealed compartment approach, including, for example, sealed blister-packs or covered compartments. Compartments can be opened (for example by mechanical action) to initiate evaporation. For example, a blister-pack containing a volatile material can be placed in a lid/vessel assembly that incorporates protrusions inside the cavity, and turning or otherwise actuating the lid can cause these protrusions to squeeze or puncture the blister-pack and release the volatile material, initiating evaporation. A volatile material can be stored in a sealed compartment. The compartment can be placed inside a vapor pressure pump comprising a lid and vessel (see, e.g., FIG. 7A). This vapor pressure pump can be pre-assembled. The sealed compartment containing the volatile material can be achieved using a combination of lid and vessel. The design of this lid and vessel can be such that motion of the lid or another structure (e.g., a cover) connects or disconnects the contents of the sealed compartment with the air space of the vessel. In addition, the vessel can be divided to contain a volatile material and one or more separate sample compartments.

Once released, the volatile material can evaporate into a chamber (see, e.g., FIG. 7B). The chamber can be isolated from the atmosphere, so evaporation of the volatile material can increase the pressure in the chamber. Once the volatile material reaches equilibrium with its vapor, the pressure can be higher than the atmospheric pressure, and its value can be calculated using a thermodynamic vapor equilibrium model, such as described herein.

During pumping, evaporation of additional liquid can provide additional pressure, although there is a drop in pressure, since the volume previously occupied by sample is now available to the gas phase, effectively causing expansion. This pressure drop can often be neglected, if the sample volume being pumped is much smaller than the pump gas compartment volume. Once the entire sample has been pumped through the device, the vapor in the lid can be connected to the atmosphere in which case the gauge pressure will drop.

This method of vapor pressure pumping can be used independently or in conjunction with compression (such as the pumping lid approach described herein). When generating positive pressure, the compression can be used to increase the range of pressures that can be achieved with the vapor pressure approach. In the case of gas expansion, the use of vapor pressure can set a lower limit to the pressure that can be obtained to the vapor pressure of the volatile material.

Because vapor pressure is a function of temperature (see, e.g., Eq. 14 and Eq. 15) the equilibrium pressure of a volatile material/air system can increase with temperature. The change in pressure with temperature can exceed the change predicted for heating of an ideal gas in a closed volume. Heaters (e.g., microfabricated heaters) or other thermal control systems can thus be used to precisely control the pressures provided by this pump. Heating a volatile material can be used to generate a pressure within a chamber, alone or in combination with other pressure generation techniques described herein.

FIG. 7A illustrates an example of generation of pressure using volatile material equilibrium. FIG. 7A is a schematic of parts that can be used for volatile material equilibrium pressure generation with a combination of lid and vessel. Compartments 700 can be used to store samples and are part of a vessel. Guiding structure 705 can engage with docking structure 730. The samples can be loaded via hole 710, the handles 735 can be used for actuation of the lid, which can consist of a nozzle for pressure measurement 720 and made of a material 715. FIG. 7B illustrates one example of a method that can be used to generate pressure with a combination of lid and vessel. The figure shows the cross section of the lid 741 and vessel 743 assembly on the device 744. Prior to the experiment, a volatile material 742 and the sample 745 can be placed in isolated compartments of the cup. At this stage, the pressure in the lid cavity is equilibrated with the atmosphere. When the lid is rotated in step 750, the volatile material is exposed to the air in the cavity and starts to evaporate in step 751 to reach its equilibrium pressure, thus starting pumping step 755. Evaporation in step 756 continues as pumping in step 757 begins. The pumping is complete in step 760 and the cavity is in contact with the external atmosphere and the pressure returns to zero. FIG. 7C illustrates an experimental pressure profile obtained by performing the steps described in FIG. 7B, for pumping 20 μL of water. Evaporation is started at point 770, equilibrium pressure is reached at point 771, pumping is started at point 772, pumping proceeds at point 773, and pumping is complete at point 774. FIG. 7D gives the pressure profile obtained when pumping a 2 mL sample volume through a microfluidic device. FIG. 7E gives equilibrium pressures obtained by using mixtures of liquids at different molarities. The dashed line indicates the linear fit of the data and its parameters are reported in the graph. FIG. 7F gives equilibrium pressure obtained using a sample at different temperatures.

### Springs, Magnets, and Elastic Pressure Vessels

A pumping lid can be moved or actuated (e.g., up or down to expand or compress a cavity) by a force. Such forces can be applied manually by a user. Alternatively, such forces can applied by, for example, magnetic, elastic, or spring forces. Applied forces can guide the motion of a pumping lid with or without the use of a guiding structure. Motion caused by a force such as magnetic, elastic, or spring forces can be held back by a lock, clasp, or other suitable structure. Once the motion of the lid is desired, the structure can release and allow the forces to move the lid. The forces can be released by mechanical actions, such as turning the lid and releasing locks that kept the lid in place.

In one example, one magnet can be attached to the pumping lid while another magnet can be positioned underneath the vessel. Upon placement of the pumping lid onto the vessel, both magnets will attract each other and pull the pumping lid down, generating positive gauge pressure. The speed with which positive pressure is generated can be tailored, for example by tuning the magnetic forces or the frictional forces. In another example, repulsive magnetic forces can be used to push the pumping lid away from a vessel to generate negative pressure for liquid actuation.

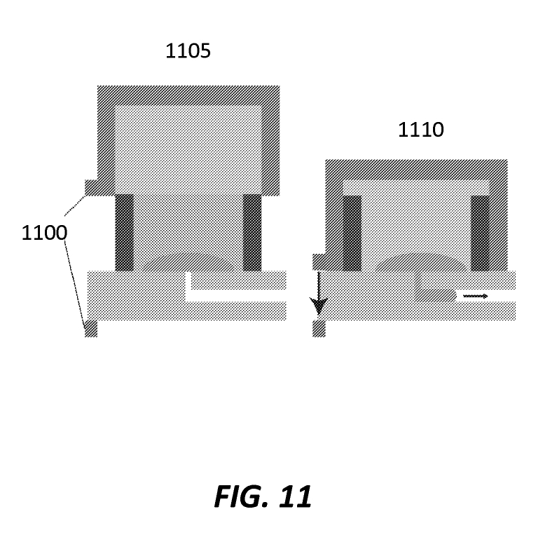

FIG. 11 provides a schematic representation of the generation of positive pressure using magnetic force. Magnets 1100 can be placed on the pumping lid and vessel, so that both magnets will attract each other and pull the pumping lid down from FIG. 1105 to FIG. 1110, thus starting pumping of the sample. In another embodiment, repulsive magnetic forces can be used to push the pumping lid away from the vessel to generate negative pressure for liquid actuation.

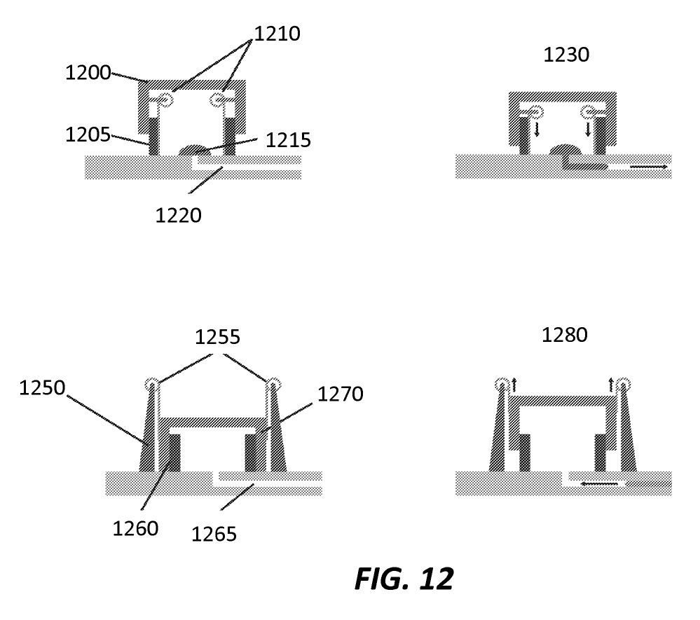

The force needed for pushing the lid and bringing it to its final position can be generated using the energy stored in one or more objects, such as, for example, a stretched or compressed elastic object or material, such as a spring or band. Springs can include regular linear springs or constant force springs. The movement of the lid produced with this technique can result in gas compression (thus generation of positive pressure) and/or gas expansion (thus generation of negative pressure). FIG. 12 shows examples of generation of positive (top) and negative (bottom) pressure using two springs as the driving force for pumping.

FIG. 12 provides examples of generation of positive and negative pressure using two springs as the driving force for the pumping lid. Positive pressure onto sample 1215 can be generated with pumping lid 1200, vessel 1205 and springs 1210, so that the sample can travel into the fluidic channel 1220, as shown in scheme 1230. Negative pressure onto a sample can be generated with pumping lid 1270, vessel 1260, springs 1255 and support for springs 1250, so that the sample can travel from fluidic channel 1265 into the vessel, as shown in scheme 1280.

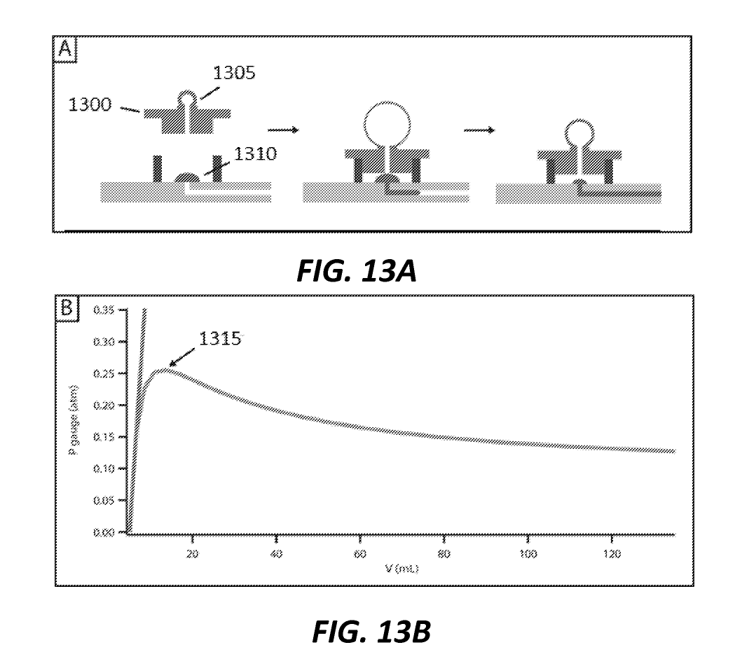

FIG. 13A provides an example of elastic deformation of a spherical vessel for pressure stabilization during pumping. An elastic element 1305 can be attached to pumping lid 1300, placed onto a vessel filled with a sample 1310 (see, e.g., FIG. 13A). As pumping proceeds, the elastic is expanded. As the elastic is compressed, the sample can flow back out via the fluidic channel. FIG. 13B graphs the gauge pressure inside a spherical elastic element as a function of its equibiaxial deformation.

Potential energy may be stored in a material (such as an elastic material) during positive or negative gauge pressure generation. The walls of the vessel and/or lid can be made of an elastic material that can be deformed upon application of pressure. When a non-zero gauge pressure is created (for example, positive pressure generation by placing the lid on the vessel) the elastic material can absorb some of the energy by stretching (see, e.g., FIG. 13A). In one example, the overall pressure can thus be lower than expected from a rigid pumping lid/vessel system of the same dimensions. As pumping proceeds, the restoration of the elastic parts (of the lid, vessel or both lid and vessel) to its original shape can reduce the expansion/compression that the gas is undergoing in the cavity as liquid is pumped in/out, thereby limiting the variation in pressure. For example, positive pressure can be generated by this method in the initial region of stretching of the material (where the energy of the material can be modeled by Hooke's law), and/or in the region of more intense stretching, post “snap buckling” regime (see, e.g., FIG. 13B). Such deformation of elastic material can accurately be represented by the Ogden model. After the material “snaps through” at a certain deformation, the pressure decreases as the material keeps deforming (deformation can be achieved by pumping, for example). The effect of snap buckling can be used to offset the pressure decrease due to pumping, thereby providing a more stable source of pressure and flow rate. This method can be beneficial because it is can increase the pressure during pumping if operated in the post “snap buckling” regime.

### Venting and Contamination-Free Pressure Equilibration

It can be desirable to equilibrate the pumping lid with atmospheric pressure at a certain point during operation. For example, equilibration of the pressure within the lid to atmospheric pressure can be used to terminate pumping or to allow for sample storage. Venting can be also useful during steps that involve temperature changes (e.g., thermocycling, incubation, storage, climate conditions), since thermal expansion/contraction may alter the pressure in the system. In addition to via holes described herein, other methods can be used to facilitate venting. For example, a small section of the pumping lid can be made of soft material which can be punctured with a needle (such as a septum cap, for example e3D printed as a single multi-material part). If the pumping cup is bonded to the device in a detachable way, another method to depressurize the system can be to simply disconnect the cup from the inlet.

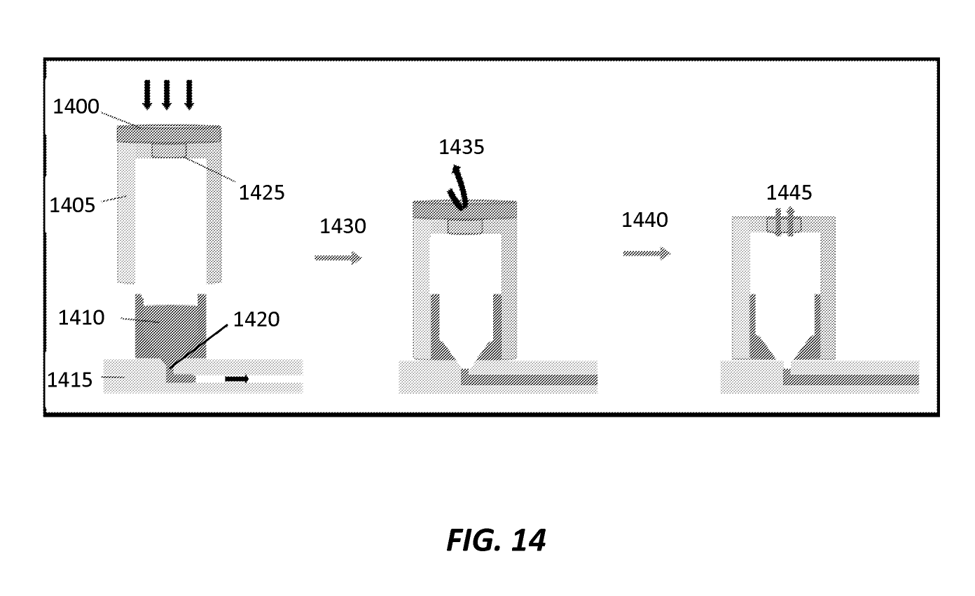

Pumping lid designs can be used to reduce or eliminate contamination. This can be useful, for example, when handling potentially infectious or otherwise dangerous samples, or when handling samples that are sensitive to contamination from surroundings or have potential to contaminate the surroundings. A trap, such as an aerosol filter, can be used as part of the pumping lid to avoid contents from escaping or outside contamination from getting inside (one non-limiting example is shown in FIG. 14). This filter can permit pressure equilibration with ambient pressure, while acting as a two-way barrier for contamination prevention. Exposure of the filter can be performed in a manual or an automated way (e.g. peeling of foil, opening a lid, opening a valve, breakable rigid cover initiated by slipping, breakable rigid cover initiated by thermal step). Exposure of the filter can be conducted, for example, after pressurization steps and before any thermal steps (e.g., thermocycling).

A schematic of one example of this approach using positive pressure is shown (FIG. 14); this method can be applicable for negative pressure pumping as well. FIG. 14 shows an exemplary schematic of a pumping lid 1405 with a filter 1425 to prevent contamination and a seal 1400 covering the filter. A sample can be loaded into an inlet 1420. The lid 1405 can be placed on a vessel 1410 that is on the device 1415, and used to compress the chamber to increase pressure and begin sample loading. After loading is complete, the microfluidic device may or may not be slipped, depending on the application. The seal, which can be rigid or flexible, can be removed in step 1435, which depressurizes the inner chamber. Depressurization can halt pumping and allow for manipulations such as a thermocycling step 1440. In this example, the filter prevents contamination from entering the system but allows gas exchange of the sample aerosols in step 1445.

A method to prevent aerosol generation and contamination can be to cover an aqueous sample with a light-weight oil of lower density than the sample, thereby minimizing sample exposure and opportunity for contamination/aerosol generation. Oil can be stored as part of the pumping lid and released before, during, or after pumping. Various approaches (e.g. described herein for multi-step manipulations) can be used herein to induce release of the oil.

### Pumping Vessel Attachment

A pumping vessel can be attached to a device (e.g., a fluidic device, a microfluidic device) in either a permanent or detachable way. Attachment can be achieved using double-sided tape, UV-curable epoxy, glue, magnets placed on opposite sides of the device, tightening latches, a number of clamping mechanisms, or other methods.

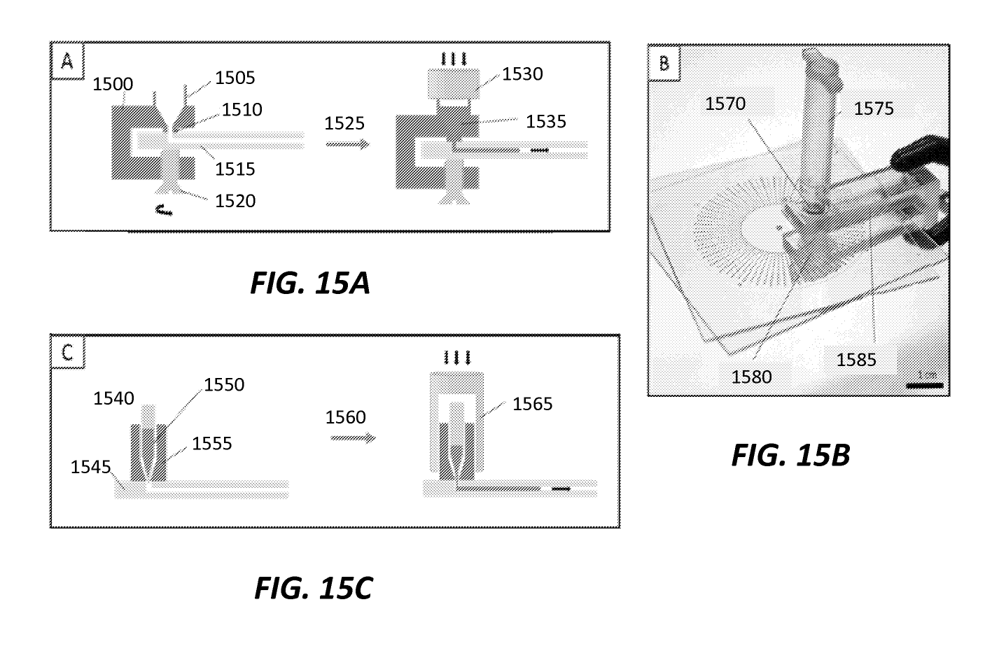

To demonstrate one example of clamping, a 3-D printed integrated pumping vessel and C-clamp design was used to load a microfluidic (e.g., SlipChip) device (FIG. 15A). A C-clamp can provide a clamping force to hold together the SlipChip and reduce the gap between the SlipChip plates. A 3-D printed screw can hold the pumping vessel to a SlipChip device. Other tightening mechanisms may be used, such as, for example, fasteners, latches, levers, magnetic clamps, bar clamps, toggle clamps, and clips.

The C-clamp design can be compatible with multiple cups for simultaneous loading through many ports. After loading, the device can be slipped and the vessel/pipette tip can be easily removed by loosening the screw and sliding the C-clamp attachment off the device. In some instances, it is desirable to reuse the C-clamp, but to dispose of the pumping vessel. To address this, the pumping vessel can be designed to attach and detach from the C-clamp, allowing for disposal of the pumping cup after use.

In some instances, it is desirable to slip the SlipChip device while maintaining the connection between the vessel and the device inlets. The integrated C-clamp pumping vessel can be designed to slip selectively with either the top or bottom plate. One way this can be achieved is by using a soft material (e.g., to achieve more friction) in contact with the glass on the top plate containing inlets and a hard material for the opposite face. In this case, during slipping, the C-clamp can stay attached to the top plate but move relative to the bottom plate. In other examples, it may be desirable to disconnect the pumping cup from the device inlet during slipping. To achieve this, for example, soft material can be used for both plates. The soft material contact area and positioning is tuned such that the integrated C-clamp pumping cup moves in coordination with the bottom plate.

FIG. 15A and FIG. 15B provide examples of a microfluidic (e.g., SlipChip) loading with an integrated C-clamp pumping lid. FIG. 15A shows a schematic outline of the steps, wherein the integrated C-clamp pumping lid is placed over the ports and attached by applying pressure with a screw. The sample is then placed into the pumping cup and the pumping lid is pressed on top to apply pressure, thereby loading the sample. FIG. 15B shows a photograph of an exemplary multivolume SlipChip device that can be used for digital nucleic acid quantification loaded with the integrated C-clamp pumping lid. FIG. 15C shows an exemplary schematic of pipette tip loading in conjunction with the pumping lid. The cup and lid can be designed to accommodate the volume of the pipette tip. The tip can be loaded with sample and inserted into the vessel. The lid can then be inserted over the vessel and pressure is applied to facilitate loading. In addition to a screw, alternative means of attaching the C-clamp can be used.

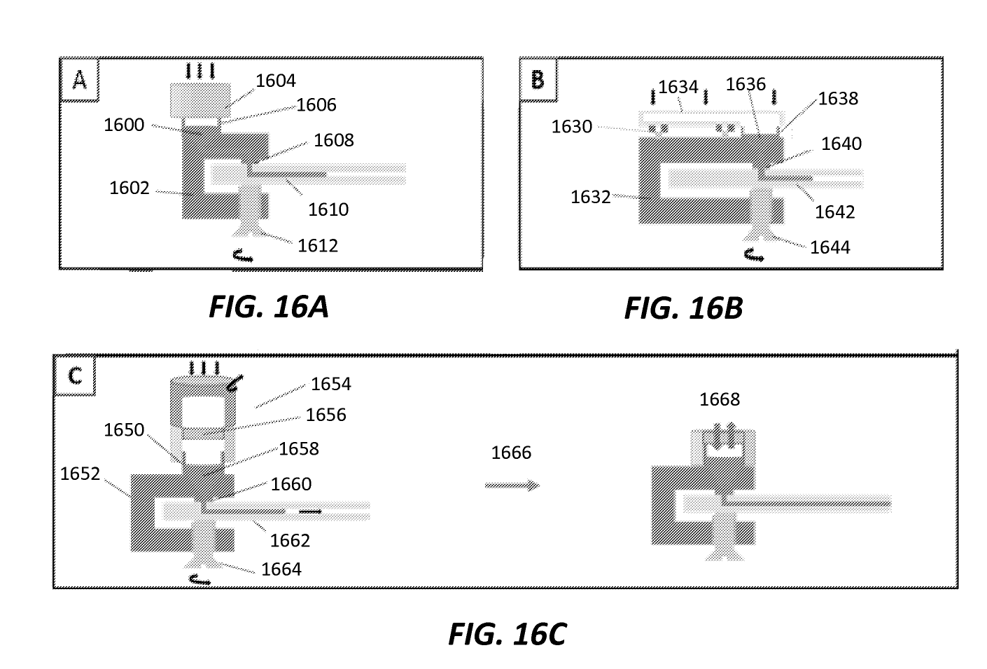

For some applications, it can be desirable to alter the position, configuration and design of the pumping lid, pumping vessel, and other components (e.g., a C-clamp). For example, it may be desirable to fit specific dimension criteria, or to avoid shadows over a readout zone. In some cases, rather than being at the end of the C-clamp just above the port, the pumping vessel can be positioned on the other end of the top arm just above the bend in the C-clamp (see, e.g., FIG. 16A). A channel within the arm of the C-clamp can then connect the pumping vessel to the device port. A sloping, horizontal, or other special configuration can be used for the pumping lid (see, e.g., FIG. 16B). A pumping lid can include a docking (e.g. snapping, insert and twist, or other) mechanism for positioning with respect to the C-clamp (see, e.g., FIG. 16B). A two-piece pumping lid can be used (FIG. 16C). This can be desirable, for example, when the integrated C-clamp pumping lid is involved with multiple steps of the reaction, such as when performing loading and venting (e.g., for thermocycling). One of the pieces of the pumping lid can house a breathable membrane or barrier (e.g., aerosol filter) while the other vessel defines the volume of the pumping lid when intact. After device loading, the other piece of the pumping lid can be detached. The design elements described herein can be used together in the combinations described, or individually, or in alternative combinations. For example, breathable membranes and two-piece pumping lids could be used in combination with a C-clamp, or in combination with other lid and clamp designs.

FIG. 16A-FIG. 16C show examples of designs of an integrated pumping vessel, pumping lid, and C-clamp. In FIG. 16A, a pumping vessel 1606 is placed over the arm of the C-clamp 1602 rather than directly over the port. A sample 1600 can be placed in the vessel 1606, and the lid 1604 can be placed on the vessel. A screw 1612 can hold the C-clamp to the device 1610. A section of the device can be made of soft material 1608, for example to improve sealing. A channel within the arm of the C-clamp connects the pumping cup to the device port. While a channel with 90 degree turns is shown, it should be clear that a straight channel can also be used. FIG. 16B illustrates a horizontal pumping vessel and docking mechanism to the C-clamp. The lid 1634 can be held in position by a structure 1630. The sample 1636 can be placed in the vessel 1638. The C-clamp 1632 can be held in place to the device 1642 by a screw 1644. A section of the device can be made of soft material 1640. The shallow design of the pumping device can reduce shadow formation.

FIG. 16C shows an exemplary two-piece pumping lid with a filter. The filter can be used for thermocycling or venting, for example. The two-piece lid 1654 can be placed on top of filter 1656, which can be on top of the vessel 1650. The C-clamp 1652 can be held in place to the device 1662 by a screw 1664. A section of the device can be made of soft material 1660. In step 1666, the lid can be removed and thermocycling can be conducted. Gas can be exchanged in step 1668 and the pressure can equilibrate.

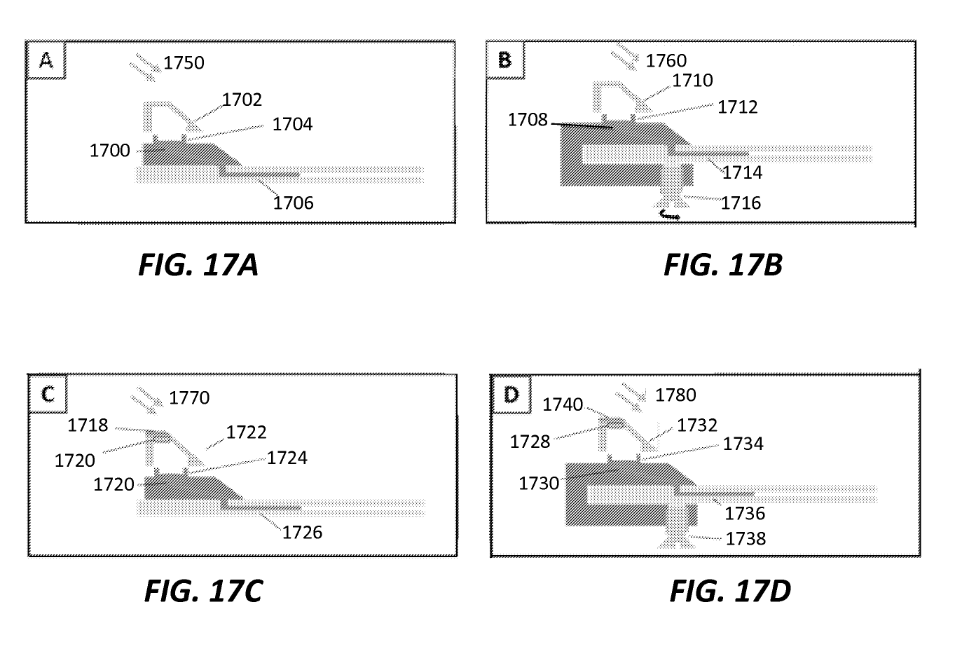

The pumping lid/vessel combination can be located away from the device wells or other regions of interest (e.g., to avoid obstructing for illumination or imaging purposes). The channel inside the lid can be made without curves, to simplify manufacturing. This design is compatible with clamping/venting techniques. By slanting the lid, illumination or imaging can be conducted from an angle relative to the device without the lid or vessel obstructing the optical path. FIG. 17A-FIG. 17D show additional variant designs of the pumping lid. FIG. 17A shows an exemplary pumping lid with a sloping profile, which can be advantageous to minimize the shadow from the pumping lid when angled illumination is used. The sample 1700 can be placed into the vessel 1704 of device 1706, and lid 1702 can be placed on top. Illumination 1750 can be present. FIG. 17B shows an exemplary pumping lid with a sloping profile, which can be useful with illumination 1760. A sample 1708 can be placed in vessel 1712, and a lid 1710 can be used to pump the sample into a device 1714. A fastener 1716 can be used to hold the pumping device in place. FIG. 17C shows an exemplary device with a sloping profile, which can be useful with illumination 1770. A sample 1720 can be placed in vessel 1724, and a lid 1722 can be used to pump the sample into a device 1726. A seal 1718 and filter 1720 can be used to prevent contamination of the sample. FIG. 17D shows an exemplary device with a sloping profile, which can be useful with the optional illumination 1780, and wherein sample 1730 can be placed in vessel 1734, and lid 1732 can be used to pump the sample into the device 1736. A seal 1740 and filter 1728 can be used to prevent contamination of the sample, and a fastener 1738 can be used to hold the pumping device in place.

For some applications, sterility and adsorption to surfaces by materials (e.g., proteins, nucleic acids, and cell adhesion) can be a concern. To provide sterility, the pumping cup and pumping lid can be bleached, autoclaved, irradiated, or decontaminated through some other standard cleaning method. To reduce or prevent adsorption, the surface of the pumping cup can be chemically modified or coated with a non-stick solution. One method that can be used is to load a low-binding material to hold the sample, such as a pipette tip (FIG. 15C). A pipette can be used to load a sample into the tip, the tip can be removed without loss of sample, and the pipette tip can be inserted into the pumping vessel and into the port. The pumping vessel and pumping lid can be designed to accommodate the pipette tip and loading. The volume of the pipette tip can be known, or can be provided by manufacturer), so the pressure can be accurately predicted. The pipette tip can also be used in conjunction with other features such as the integrated C-clamp pumping lid.

### Compartments

A device can include a plurality of vessels, where each vessel may be the same or different. Furthermore, a plurality of arrays of such vessels can be present in one or more layers of a device (e.g., arrays that can be connected sequentially or serially). Such vessels can include any volumetric structure. Vessels in a layer or an array may have the same surface dimension, cross-section, planarity, or surface characteristic. Alternatively, vessels in a layer or an array may have different surface dimensions, cross-sections, planarity, or surface characteristics. Exemplary vessels include an open groove or trench, a closed channel, or an open or closed well. Such vessels can be useful for holding or transporting one or more reagents, samples, or fluids (e.g., a lubricant). The device can include 1, 2, 3, 4, 5, 6, 7, 8, 9, 10, or more vessels.

Vessels, cavities, compartments, and other regions can be characterized by a volume. Such regions can have the same volume, or different regions can have different volumes. The volume of a vessel, a cavity, a compartment, or other region can be at least about 1 nanoliter (nL), 2 nL, 5 nL, 10 nL, 20 nL, 30 nL, 40 nL, 50 nL, 60 nL, 70 nL, 80 nL, 90 nL, 100 nL, 150 nL, 200 nL, 250 nL, 300 nL, 350 nL, 400 nL, 450 nL, 500 nL, 600 nL, 700 nL, 800 nL, 900 nL, 1 microliter (μL), 2 μL, 5 μL, 10 μL, 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, 1 milliliter (mL), 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, or 50 mL. The volume of a vessel, a cavity, a compartment, or other region can be at most about 50 milliliter (mL), 45 mL, 40 mL, 35 mL, 30 mL, 25 mL, 20 mL, 15 mL, 10 mL, 9 mL, 8 mL, 7 mL, 6 mL, 5 mL, 4 mL, 3 mL, 2 mL, 1 mL, 900 microliter (μL), 800 μL, 700 μL, 600 μL, 500 μL, 400 μL, 300 μL, 200 μL, 100 μL, 90 μL, 80 μL, 70 μL, 60 μL, 50 μL, 40 μL, 30 μL, 20 μL, 10 μL, 5 μL, 2 μL, 1 μL, 900 nanoliter (nL), 800 nL, 700 nL, 600 nL, 500 nL, 450 nL, 400 nL, 350 nL, 300 nL, 250 nL, 200 nL, 150 nL, 100 nL, 90 nL, 80 nL, 70 nL, 60 nL, 50 nL, 40 nL, 30 nL, 20 nL, 10 nL, 5 nL, 2 nL, or 1 nL. The volume of a vessel, a cavity, a compartment, or other region can be about 1 nanoliter (nL), 2 nL, 5 nL, 10 nL, 20 nL, 30 nL, 40 nL, 50 nL, 60 nL, 70 nL, 80 nL, 90 nL, 100 nL, 150 nL, 200 nL, 250 nL, 300 nL, 350 nL, 400 nL, 450 nL, 500 nL, 600 nL, 700 nL, 800 nL, 900 nL, 1 microliter (μL), 2 μL, 5 μL, 10 μL, 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, 1 milliliter (mL), 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, or 50 mL.

### Channels

A device can comprise one or more channels, conduits, and ports (e.g., inlets or outlets). The channel, conduit, port, inlet, or outlet can include any useful cross-section or plurality of cross-sections along their lengths. Cross-sections can be of any useful shape (e.g., rectangular, square, circular, oval, trapezoidal, triangular, or irregular cross-sections). Cross-section shape or dimensions can vary along the axis of any structure. For instance, the cross-section along the axis of fluid flow can change from one cross-sectional shape or area to another, such as from a circular to a rectangular cross-section. In another instance, the dimensions of the cross-section can be uniform or can vary along any axis, such as a conduit that tapers or expands along the axis of fluid flow.

The path of any channel, conduit, port, inlet, or outlet can be linear, twisting, curved, serpentine, or any other track shape. Twisting or serpentine passages may be selected to encourage mixing of components of a fluid. The channel, conduit, port, inlet, or outlet can additionally contain columns, posts, dimples, humps, weirs, hydrophobic patches, hydrophilic patches, or other structures to improve mixing of fluids as they pass. Implementations in which the channel, conduit, port, inlet, or outlet is linear can achieve rapid transfer of a fluid under minimal pressure. The channel, conduit, port, inlet, or outlet can be substantially axially aligned, with the upstream opening being directly or nearly directly above the downstream opening. Alternatively, the upstream and downstream opening can be offset by any distance.

A channel, conduit, port, inlet, or outlet can have a cross-sectional area of at least about 1, 2, 5, 10, 20, 50, 100, 200, 500, 1000, 2000, 5000, 10000, 20000, 50000, or 100,000 square micrometers, or 1, 10, 20, 30, 40, 50, 60, 70, 80, 90, 100, 200, 300, 400, 500, 600, 700, 800, 900 or 1000 square millimeters. A channel, conduit, port, inlet, or outlet can have a cross-sectional area of about 1, 2, 5, 10, 20, 50, 100, 200, 500, 1000, 2000, 5000, 10000, 20000, 50000, or 100,000 square micrometers or 1, 2, 3, 4, 5, 6, 7, 8, 9, 10, 20, 30, 40, 50, 60, 70, 80, 90, or 100 square millimeters.

The device can include at least 1, 2, 3, 4, 5, 6, 7, 8, 9, 10, or more channels, conduits, port, inlets, and outlets.

For channels, conduits, port, inlets, or outlets, the size, length, cross-sectional area, other geometric factors, or any combination thereof can be selected to control flow rates, pressures, or other characteristics of fluid flow.

### Fabrication and Assembly

Devices described herein (e.g., vessels, caps) can be produced as a standalone part and then connected to a separate device (e.g., a microfluidic device) by methods such as adhesive bonding, solvent bonding, or mechanical interlocking. Alternatively, the device can be designed as an integral part of a separate device (e.g., a microfluidic device) at the time the device is produced. Devices described herein can comprise various components, including a lid, a docking structure, and a guiding structure that engages with at least one docking structure.

The device or components thereof can be formed by any useful process, including but not limited to molding (e.g., injection molding, vacuum molding, or over-molding), machining (e.g., drilling, milling, or sanding), embossing (e.g., hot embossing) and etching (e.g., laser, deep reactive ion etching, KOH etching, or HF etching). In microfluidic applications, the layers can be fabricated from a material that enables formation of high resolution features such as microchannels, chambers, mixing features, and the like, that are of millimeter, micron, or submicron dimensions (e.g., PDMS, PMMA, glass). Applicable microfabrication techniques include but are not limited to dry etching, wet etching, laser etching, laser ablation, molding, embossing, photolithography, soft lithography, lamination or the like. Multi-material 3D printing (e.g., using a Objet 260 system, Stratasys, Eden Prairie, Minn., USA) can be used, which can produce parts composed of two different materials, and mixtures of these two materials.

### Materials

The device can be made of low-cost, disposable/recyclable polymeric materials. The device may be useful in resource-limited settings.

The device, lid, guiding structure, docking structure, docking pin, chamber, or other structure can be formed from any useful material. The materials used to form the devices of the invention are selected with regard to physical and chemical characteristics that are desirable for proper functioning of the device. Suitable, non-limiting materials include polymeric materials, such as silicone polymers (e.g., polydimethylsiloxane and epoxy polymers), polyimides (e.g., commercially available Kapton® (poly(4,4′-oxydiphenylene-pyromellitimide, from DuPont, Wilmington, Del.) and Upilex™ (poly(biphenyl tetracarboxylic dianhydride), from Ube Industries, Ltd., Japan)), polycarbonates, polyesters, polyamides, polyethers, polyurethanes, polyfluorocarbons, fluorinated polymers (e.g., polyvinylfluoride, polyvinylidene fluoride, polytetrafluoroethylene, polychlorotrifluoroethylene, perfluoroalkoxy polymer, fluorinated ethylene-propylene, polyethylenetetrafluoroethylene, polyethylenechlorotrifluoroethylene, perfluoropolyether, perfluorosulfonic acid, perfluoropolyoxetane, FFPM/FFKM (perfluorinated elastomer [perfluoroelastomer]), FPM/FKM (fluorocarbon [chlorotrifluoroethylenevinylidene fluoride]), as well as copolymers thereof), polyetheretherketones (PEEK), polystyrenes, poly(acrylonitrile-butadiene-styrene) (ABS), acrylate and acrylic acid polymers such as polymethyl methacrylate, and other substituted and unsubstituted polyolefins (e.g., cycloolefin polymer, polypropylene, polybutylene, polyethylene (PE, e.g., cross-linked PE, high-density PE, medium-density PE, linear low-density PE, low-density PE, or ultra-high-molecular-weight PE), polymethylpentene, polybutene-1, polyisobutylene, ethylene propylene rubber, ethylene propylene diene monomer (M-class) rubber), and copolymers thereof (e.g., cycloolefin copolymer); ceramics, such as aluminum oxide, silicon oxide, zirconium oxide, and the like); semiconductors, such as silicon, gallium arsenide, and the like; glass; metals; as well as coated combinations, composites (e.g., a block composite, e.g., an A-B-A block composite, an A-B-C block composite, or the like, of any materials described herein), and laminates (e.g., a composite material formed from several different bonded layers of identical or different materials, such as polymer laminate or polymer-metal laminates, e.g., polymer coated with copper, a ceramic-in-metal or a polymer-in-metal composite) thereof.

The weight of the device may be light, and thus, the device may be portable. The total weight of the device may be about 1000 grams (g), 750 g, 500 g, 400 g, 300 g, 200 g, 100 g, 90 g, 80 g, 70 g, 60 g, 50 g, 40 g, 30 g, 20 g, or 10 g. The total weight of the device may be less than 1000 grams (g), 750 g, 500 g, 400 g, 300 g, 200 g, 100 g, 90 g, 80 g, 70 g, 60 g, 50 g, 40 g, 30 g, 20 g, or 10 g. In some examples, the combined weight of all parts in a device was less than 50 g.

Integration with Other Devices

Pumping lids and vessels can be integrated with other devices, such as to provide pressure or to allow multistep processes. Examples include but are not limited to devices for multistep protocols for nucleic acid extraction and filtration elements to separate plasma from whole blood using membranes and/or integrated filtration elements such as geometrical features in the device (for example, restrictions or a gap between the plates).

The present pressure control devices and methods can be integrated with any useful device, such as a fluidic device. These fluidic devices can include multiple substrates or layers. The pressure control devices can be integrated with a fluidic device (e.g., a microfluidic device, a SlipChip device), or with any type of device having any useful structure. The present pressure control system can be integrated with any device by providing fluidic connections between the components of this system with a chamber within a fluidic device. The pressure control device can be integrated with the device, so the method may not require the use of external connectors or tubing.

Furthermore, fluidic devices can be integrated with another device. For example, a first fluidic device for nucleic acid sample preparation can be integrated with a second fluidic device for amplification, where the first device is fluidically connected to the second device, and the pressure control system is fluidically connected to the first device and/or the second device. In yet another example, the functionalities that can be performed in two or more fluidic devices can be built into a single, multi-structured fluidic device (e.g., a device having multiple substrates, where each functionality occurs in a separate substrate, or a device having multiple sections, where each functionality occurs in a separate section).

A fluidic device can be a microfluidic device, such as a SlipChip device. A SlipChip device can comprise one or more layers that allow for connection and disconnection of one or more chambers by relative movement. For example, in a first position, a first chamber is not connected to a second chamber (i.e., the first chamber does not fluidically communicate with the second chamber). Upon moving the first chamber relative to the second chamber, a connection is formed. This movement can be accomplished by moving the first layer having the first chamber relative to the second layer. Alternatively, this movement can include moving the second layer having the second chamber relative to the second layer. The connection between chambers can also occur via a capture region, a bridge, a membrane, or any other structure described to provide fluidic communication between a first and second chamber. This SlipChip platform can be fabricated from a variety of materials, such as glass and plastic. A plastic rotational SlipChip with user friendly features manufactured using 3D-printing was previously demonstrated. A user simply loads the sample into the sample chamber, closes the lid to apply pressure, holds the bottom disc, and rotates the top portion to perform sample preparation. The pumping lid can be used to load SlipChip devices using either positive or negative pressures. This may be useful because loading SlipChip devices require control of the inlet pressure within a defined range, and SlipChips can be used in limited resource settings (LRS) by untrained users.

In some examples, a fluidic device (e.g., a SlipChip device) can be loaded by negative pressure with a pumping lid device described herein. A fluidic device designed for multivolume digital nucleic acid amplification may be used, which can present challenges in filling due to variation of capillary pressure among wells of different sizes. Previously, this type of device was filled by positive pressure and dead end filling. In this example, the device is modified for negative-pressure filling by addition of a sealing ring filled with high-vacuum grease (sealing structure) around the active area containing the amplification wells (FIG. 6B). An outlet for oil to the device may be added, over which the negative-pressure pumping lid was placed. The device can be assembled such that the lubricating oil (e.g., 5 cSt silicone oil) fills the wells. For loading, a sample of 50 μL of 0.5 M FeSCN aqueous solution can be placed onto the inlet, and the pumping lid can be pulled up to create negative pressure of 0.1 atm, remove excess oil and draw the sample into all of the wells of the device (FIG. 6B). This experiment demonstrates that bubble-free filling can be accomplished using the pumping lid, and that complex devices (a combination of immiscible fluids and wells with different capillary pressures) can be handled.

A fluidic device can include one or more substrates, layers, chambers, capture regions, or other structures having any useful dimension. Useful dimensions include any length, width, or depth that can be uniform or varied along any useful axis. Exemplary dimensions in any useful axis (e.g., perpendicular to the axis of fluid flow) include less than about 50 mm (e.g., less than about 40 mm, 20 mm, 15 mm, 10 mm, 5 mm, 2 mm, 1 mm, 500 μm, 200 μm, 60 μm, 50 μm, 40 μm, 30 μm, 15 μm, 10 μm, 3μm, 1 μm, 300 nm, 100 nm, 50 nm, 30 nm, or 10 nm), or from about 10 nm to about 50 mm (e.g., 10 nm to 40 mm, 10 nm to 20 mm, 10 nm to 15 mm, 10 nm to 10 mm, 10 nm to 5 mm, 10 nm to 2 mm, 10 nm to 1 mm, 10 nm to 500 μm, 10 nm to 200 μm, 10 nm to 60 μm, 10 nm to 50 μm, 10 nm to 40 μm, 10 nm to 30 μm, 10 nm to 15 μm, 10 nm to 10 μm, 10 nm to 3μm, 10 nm to 1 μm, 100 nm to 50 mm, 100 nm to 40 mm, 100 nm to 20 mm, 100 nm to 15 mm, 100 nm to 10 mm, 100 nm to 5 mm, 100 nm to 2 mm, 100 nm to 1 mm, 100 nm to 500 μm, 100 nm to 200 μm, 100 nm to 60 μm, 100 nm to 50 μm, 100 nm to 40 μm, 100 nm to 30 μm, 100 nm to 15 μm, 100 nm to 10 μm, 100 nm to 3 μm, 100 nm to 1 μm, 1 μm to 50 mm, 1 μm to 40 mm, 1 μm to 20 mm, 1 μm to 15 mm, 1 μm to 10 mm, 1 μm to 5 mm, 1 μm to 2 mm, 1 μm to 1 mm, 1 μm to 500 μm, 1 μm to 200 μm, 1 μm to 60 μm, 1 μm to 50 μm, 1 μm to 40 μm, 1 μm to 30 μm, 1 μm to 15 μm, 1 μm to 10 μm, 1 μm to 3 μm, 10 μm to 50 mm, 10 μm to 40 mm, 10 μm to 20 mm, 10 μm to 15 mm, 10 μm to 10 mm, 10 μm to 5 mm, 10 μm to 2 mm, 10 μm to 1 mm, 10 μm to 500 μm, 10 μm to 200 μm, 10 μm to 60 μm, 10 μm to 50 μm, 10 μm to 40 μm, 10 μm to 30 μm, 10 μm to 15 μm, 50 μm to 50 mm, 50 μm to 40 mm, 50 μm to 20 mm, 50 μm to 15 mm, 50 μm to 10 mm, 50 μm to 5 mm, 50 μm to 2 mm, 50 μm to 1 mm, 50 μm to 500 μm, 50 μm to 200 μm, 50 μm to 60 μm, 100 μm to 50 mm, 100 μm to 40 mm, 100 μm to 20 mm, 100 μm to 15 mm, 100 μm to 10 mm, 100 μm to 5 mm, 100 μm to 2 mm, 100 μm to 1 mm, 100 μm to 500 μm, or 100 μm to 200 μm).

The dimensions of any structure (e.g., one or more chambers) can be chosen to maintain a particular volumetric or linear flow rate of a fluid in the device, for example while under the influence of pressure from a pumping lid. Such dimensions can be useful to control the filling of the device with particular fluids or the flow rate of such fluids through the device. The substrate, layer, chamber, capture region, or other structure can include any useful planarity. In some instances, the surfaces of the first and second layers are substantially planar to facilitate movement of these layers. Such substrates or layers can further be uniform or non-uniform in other characteristics, such as height, width, and/or depth. Alternatively, the surfaces of the structures can be non-planar and substantially complementary to allow for movement. For instance, one or more layers can include a curvilinear surface, such as the surface of a cylinder, a concave surface, or a convex surface. In one example, the first layer can include a first cylindrical surface, and the second layer includes an annular cylinder having an opening, an inner cylindrical surface, and an outer cylindrical surface. When the first layer is inserted into the opening of second layer, the first cylindrical surface and the inner cylindrical surface of the second layer are complementary, thereby allowing the first layer to move within the second layer. Accordingly, the layers can include any useful complementary surfaces, such as concentric spheres, cones, or cylinders.

For example, a pressure control device such as a pumping lid can be integrated with one or more of devices having a barrier layer, blocks configured to slide relative to each other, a sample metering channel, a cover plate, a separator for separating blood constituents in the sample liquid, a venting device, an entry port, an elongated separation chamber, one or more particles, one or more capillary passageways, one or more flow channels in combination with one or more separation means, a loading chamber, a separation chamber, a waste chamber, one or more material separation regions, one or more dispensers, one or more porous membranes including a semi-permeable barrier, one or more charge-switch nucleotide probes, one or more enrichment channels including enrichment medium, one or more storage compartments, one or more seals, one or more reaction layers having one or more reaction areas, one or more lysing chambers, one or more mixers, one or more reservoirs, one or more reaction chambers, one or more exhaust chambers, one or more enrichment columns, one or more reservoirs, one or more diaphragm valves, one or more fluid transporters, one or more flow activators, one or more actuators, one or more vacuum chambers, one or more valves, one or more gas-filled reservoirs, one or more rotatable housing members, one or more separation means, one or more temperature zones, one or more cartridges, one or more processing chambers, one or more sealing apparatuses, one or more sliders, one or more valves, and/or one or more microcapillary tubes.

### Additional Components of the System or Device

Pressure control devices, integrated fluidic devices, and other aspects of this disclosure can employ other useful components, including but not limited to air vents, electrical circuits, pressurization apparatuses, loading apparatuses, injection ports, heating elements, cooling elements, lysis components, detectors, electrodes, markers, and other elements.

Air vents can be present in a system or device. For instance, when particular assays require heating, having an open system may be useful in order to prevent pressure buildup. Accordingly, one or more air vents can be fluidically connected to a chamber in the device (e.g., one or more process chambers) that allows for access to the environment. In some cases, the air vent further includes a valve, whereby the valve can be opened to fluidically connect the air vent to the chamber. Valves can be controlled manually or automatically. A valve may be useful when reagents are provided within the device in a stored, dried, or inactivated state.

One or more valving systems can be present in the system or device. For instance, one or more valves can be included in the device to control the fluidic communication between chambers, channels, or other elements. Valves can be controlled manually or automatically.

Electrical circuits can be present in a system or device. For instance, a circuit may underlie the pressure generation system, a fluidic device, or both. In some cases, a circuit can include one or more conductive structures having junctions that can be reversibly contacted with one or more conductive materials. The electrical circuit can used to connect one or more components, including but not limited to coolers, heaters, valves, switches, power sources (e.g., batteries), sensors, detectors, communications equipment, and other components.

Any of the devices or systems herein can include electrically conductive material (e.g., one or more electrodes, including arrays thereof). Such electrodes and arrays may be useful for conducting electrochemical reactions for detection, separation (e.g., electrophoretic separation), transport, and/or synthesis. In some cases, one or more electrodes are arranged to allow for connection or disconnection upon relative movement of the layers.

Detectors can be present in a system or device. For example, imaging or sensor components can be used to record or measure reactions within a device by techniques including but not limited to optical detection, x-ray detection, absorption spectrometry, matrix-assisted laser desorption/ionization (MALDI), mass spectrometry, Raman spectrometry, fluorescence correlation spectroscopy (FCS), fluorescence polarization/fluorescence correlation spectroscopy (FP/FCS), fluorometric detection, colorimetric detection, chemiluminescence, bioluminescence, scattering, surface plasmon resonance, electrochemical detection, electrophoresis, lasers, or fluorescent imaging plate reader (FLIPR®, Molecular Devices) assays. Examples of such detectors and imaging devices can be found in U.S. Pub. No. 2009-0010804 and Int. Pub. No. WO 2008/002267, both of which are incorporated herein by reference. The detector can comprise any detector suitable to detect a signal from a device, and can be selected from the group consisting of: a web camera, a digital camera, a digital camera in a mobile phone and a video camera, for example as described in Int. Pub. No. WO 2008/002267, incorporated by reference herein in its entirety. The detector can comprise a camera or imaging device which has adequate lighting and resolution for spatially resolving individual signals produced by the device, for example as described in U.S. Pub. No. 2009-0010804, incorporated by reference in its entirety. The detector can comprise any solid state image sensor including a charged coupled device (CCD), charge injection device (CID), photo diode array (PDA), or complementary metal oxide semiconductor (CMOS). The detector can comprise a photomultiplier tube (PMT).

Markers, such as lines, dots or visible substances in ducts and/or chambers can be present in a system or device. Markers can be used to enable registration or analysis. Registration marks may be included on the device to allow for automatic correction of optical aberrations, or adjustment of the image for the angle and orientation at which the picture was taken. For detecting fluorescent output, chirped excitation/readout can be used. For example, a device can be exposed to blue excitation light for, for example, nanoseconds, then turned off, and fluorescence may be detected, for example, a nanosecond later. Then, ten nanoseconds later, for example, another image is collected (without an initial excitation flash) to produce a background intensity image for subtraction. In this manner, fluorescence can be analyzed even in daylight. For safety, the detector could be designed to recognize the device automatically, for example if the device includes a recognizable pattern, such that the detector would only produce the excitation light when pointed at the device (see, e.g., Sia et al., Angewandte Chemie Int. Ed. 43:498-502 (2004), incorporated by reference herein, which describes additional means for detecting signals in multifluidic devices, including using pulse modulation to reduce noise). Detection can also be improved by using the polarization of excited/emitted light, as is known to those skilled in the art.

Any of the devices or systems herein can be integrated with a pressurization apparatus (e.g., any described herein), a loading apparatus (e.g., any described herein), an injection port for serial or sequential filling of chambers, a heating element, an on-chip lysis component, or molecular recognition module. For instance, a device can be integrated with temperature control methods suitable for sample lysis for nucleic acid extraction, such as, temperature control methods based on simple phase transitions, where temperature is maintained constant during solid-liquid and liquid-solid phase transition, as described in the original application. As another example, a device can be integrated with on-chip initiation mechanisms for temperature control, such as initiation by relative movement (e.g., slipping) and mixing.

The devices, methods, and systems of the invention can include any number of characteristics, elements, modifications, or benefits, including but not limited to being sterile before use (e.g., the device can be assembled in a sterile environment and then packed in a sealed container until sample collection); being resistant to interference and contaminants until final analysis (e.g., a lubricant can be provided between the layers and can act as a barrier between the sample and the external world to prevents contamination and avoids leaks of potentially dangerous analytes present in the stored samples); being capable of electrical power-free usage, wherein a device or system can require no power for fluid handling (autonomous biospecimen collection) or drying (no need for heating or ventilation); being adaptable for easy digitized storage and rehydration (e.g., the device allows for precise manipulation of many volumes in parallel, where the sample can be split or partitioned into small volumes or aliquots and preserved in a digitized format, and such samples can be selectively, fully, or partially recovery for on-chip or off-chip analysis); being easy to manufacture (e.g., amenable to mass production using inexpensive materials and fabrication techniques); being modular and reconfigurable (e.g., some of these devices allow for the development of separate modules, which can be combined to produce a complete device, and each module can thus be developed separately and then integrated in the platform); being easy to use (e.g., samples can be collected by users with minimal training and without any external equipment, where necessary steps from biospecimen collection to sample preservation can be either autonomous or require minimal action from the user (e.g. slipping the plates or pushing a button)); being adaptable for various sample sizes (e.g., some of these devices allow for easy manipulation of volumes in a wide range (e.g., 1 nL-1 mL), which includes the typical volume of biospecimen collection in limited-resource settings (e.g. the amount of blood obtained from a finger prick)); being compatible with commercial dry preservation matrices or desiccants (e.g., multi-target or multi-analyte stabilization can be achieved (including for DNA, RNA, and/or proteins), for instance by using different matrices in different parts of the storage device); being upgradable with different matrices or desiccants (e.g., new matrices, desiccants, or drying agents can be easily incorporated in the platform, accommodating integration of new developments in matrix formulation); being capable of rapid drying (e.g., drying in less than 10 minutes, which arises from working at small dimensions and can be a critical issue in preserving samples sensitive to degradation); and being adaptable for sample re-collection and downstream analysis (e.g., rehydration can be easily achieved on chip in order to recover the preserved sample).

### Computer Control Systems

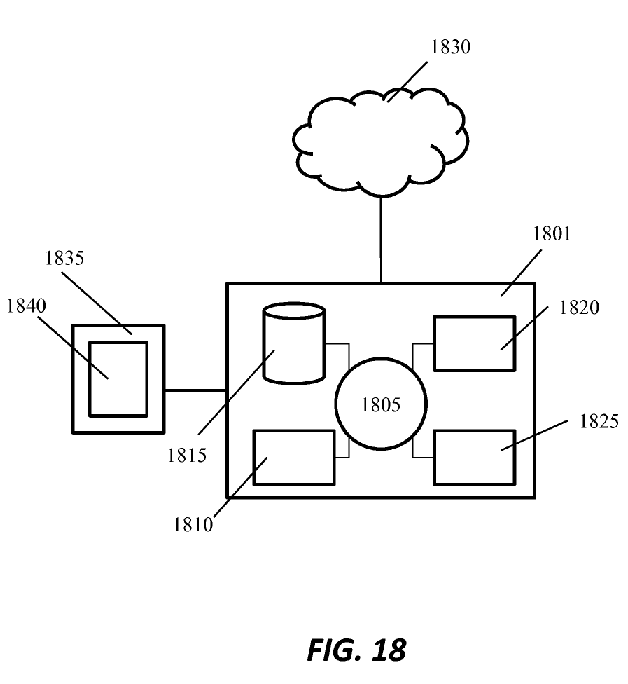

The present disclosure provides computer control systems that are programmed to implement methods of the disclosure. FIG. 18 shows a computer system 1801 that is programmed or otherwise configured to operate a device or analyze results from a device of the present disclosure. The computer system 1801 can regulate various aspects of device operation of the present disclosure, such as, for example, pumping lid motion and timing between motion steps. The computer system 1801 can be an electronic device of a user or a computer system that is remotely located with respect to the electronic device. The electronic device can be a mobile electronic device.

The computer system 1801 includes a central processing unit (CPU, also “processor” and “computer processor” herein) 1805, which can be a single core or multi core processor, or a plurality of processors for parallel processing. The computer system 1801 also includes memory or memory location 1810 (e.g., random-access memory, read-only memory, flash memory), electronic storage unit 1815 (e.g., hard disk), communication interface 1820 (e.g., network adapter) for communicating with one or more other systems, and peripheral devices 1825, such as cache, other memory, data storage and/or electronic display adapters. The memory 910, storage unit 1815, interface 1820 and peripheral devices 1825 are in communication with the CPU 1805 through a communication bus (solid lines), such as a motherboard. The storage unit 1815 can be a data storage unit (or data repository) for storing data. The computer system 1801 can be operatively coupled to a computer network (“network”) 1830 with the aid of the communication interface 1820. The network 1830 can be the Internet, an internet and/or extranet, or an intranet and/or extranet that is in communication with the Internet. The network 1830 in some cases is a telecommunication and/or data network. The network 1830 can include one or more computer servers, which can enable distributed computing, such as cloud computing. The network 1830, in some cases with the aid of the computer system 1801, can implement a peer-to-peer network, which may enable devices coupled to the computer system 1801 to behave as a client or a server.

The CPU 1805 can execute a sequence of machine-readable instructions, which can be embodied in a program or software. The instructions may be stored in a memory location, such as the memory 1810. The instructions can be directed to the CPU 1805, which can subsequently program or otherwise configure the CPU 1805 to implement methods of the present disclosure. Examples of operations performed by the CPU 1805 can include fetch, decode, execute, and write back.

The CPU 1805 can be part of a circuit, such as an integrated circuit. One or more other components of the system 1801 can be included in the circuit. In some cases, the circuit is an application specific integrated circuit (ASIC).

The storage unit 1815 can store files, such as drivers, libraries and saved programs. The storage unit 1815 can store user data, e.g., user preferences and user programs. The computer system 1801 in some cases can include one or more additional data storage units that are external to the computer system 1801, such as located on a remote server that is in communication with the computer system 1801 through an intranet or the Internet.

The computer system 1801 can communicate with one or more remote computer systems through the network 1830 (e.g., wired or wireless). For instance, the computer system 1801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1801 via the network 1830.

Methods as described herein can be implemented by way of machine (e.g., computer processor) executable code stored on an electronic storage location of the computer system 1801, such as, for example, on the memory 1810 or electronic storage unit 1815. The machine executable or machine readable code can be provided in the form of software. During use, the code can be executed by the processor 1805. In some cases, the code can be retrieved from the storage unit 1815 and stored on the memory 1810 for ready access by the processor 1805. In some situations, the electronic storage unit 1815 can be precluded, and machine-executable instructions are stored on memory 1810.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1801 can include or be in communication with an electronic display 1835 that comprises a user interface (UI) 1840 for providing, for example, pressure information, timing information, or analysis results. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1805. The algorithm can, for example, control on-device pressure, process analysis results, or operate a device.

### Samples

A device can further comprise one or more sample inlet ports or sample input wells. A sample may be loaded through a channel, conduit, inlet, or outlet. Given the pressurization inherent to the devices described herein, the sample inlet can be air tight. The sample inlet can be configured to be opened to permit addition of a sample and then resealed after the sample is loaded to the device. In one such implementation, the sample inlet can be loaded into the vessel prior to placing the lid in airtight contact with the vessel. Alternatively, the sample can be loaded via a puncturable septa or large one-way valve. The device can include an integrated sample loader, such as a bulb or syringe, useful for loading a sample into the device. The device can be packaged with a sample collection device, such as a syringe, bulb, swab, scraper, biopsy punch, or other tool for a user to collect a sample.

Samples can be obtained from a subject (e.g., human subject, animal subject), a food sample (e.g., including an organism), or an environmental sample (e.g., including one or more organisms). Exemplary, non-limiting samples include blood, plasma, serum, sputum, urine, fecal matter (e.g., stool sample), swab, sweat, spinal fluid, amniotic fluid, interstitial fluid, tear fluid, bone marrow, tissue sample (e.g., a skin sample or a biopsy sample), a buccal mouthwash sample, an aerosol (e.g., produced by coughing), nucleic acid, cell (e.g., tumor cells, fetal cells in blood, stem cells, bacterial and fungal cells, T-cells, or B-cells), protein, enzyme, soil, water, compost pile, manure pile, sediment (e.g., marine or freshwater sediment), a water sample, an air sample, rock, a plant sample, a food sample, or a gut sample. The sample can include any useful target or analyte to be detected, filtered, concentrated, and/or processed.

In some cases, all of a sample is analyzed within a device. In other cases, some of a sample (e.g., purified nucleic acid) is analyzed within a device and some is reserved for later use. In other cases, all of a sample is reserved for later use. Sample, such as purified nucleic acids, can be stored on the device or can be outlet into a sample container such as a tube or vial. A sample container can be sealed. A sample container can be sterile.

A sample can be marked, numbered, or labeled to identify its source. A mark can comprise a code, such as an alphanumeric code or an optical barcode on the device or on a sample container. A mark can comprise an electronic mark, such as data or an indicator in an RFID tag or other electronic medium. A mark can comprise unique identifiers mixed in with the sample, such as nucleic acid barcodes or particles, which can be identified later (e.g., by amplification, DNA chip readout, or sequencing). A mark, such as a nucleic acid barcode, can comprise sequencing adaptors (e.g., Illumina adaptors).

Samples can be preloaded on the device. Samples also can be loaded by a user. In some cases, some samples are provided preloaded on the device and some (e.g., perishable samples) are provided by a user prior to operation. Samples can be provided in wet or dry form. In some examples, the sample storage layer is preloaded with one or more samples. In such an example, the samples can be contained with a membrane configured to be pierced or disrupted during operation of the module. In some examples, the membrane comprises foil, laminate and/or plastic. In other examples, dry samples are rehydrated by a user prior to use of the device. For example, a user can load water into a device to rehydrate samples, and then a user can load a sample into the device and operate the device.

Exemplary samples can include, but are not limited to, lysis solutions, wash solutions, elution solutions, rehydration solutions, enzyme solutions (e.g., nucleic acid amplification enzymes, polymerase enzymes, restriction enzymes), buffers, liquid, powder, pellets, a gel, microbeads, probes, primers (e.g., primers for specific targets, such as particular organisms or infectious agents), nucleic acids, DNA, RNA, polypeptides, nucleoside triphosphates (NTPs), antibodies, a sacrificial reagent or any combination thereof. A sacrificial sample can comprise an aqueous solution, a lubricant, an oil, an aqueous-immiscible liquid, a gel, a gas, a fluorocarbon oil, a surfactant, gas, air, or any combination thereof. For example, the air can be used to generate air bubble for mixing. As another example, air and immiscible liquid can be used to remove leftover solution (dead volume) in the matrix. Samples can be mixed to change their composition. For example, one type of buffer can be mixed with another buffer or a dry reagent to change its composition to another buffer.

Exemplary infectious diseases can include, but are not limited to, actinomycosis, acquired immunodeficiency syndrome, anthrax, astrovirus infection, bacterial pneumonia, cat-scratch disease, chlamydia, cholera, coccidioidomycosis, Colorado tick fever, common cold, epidemic typhus, fatal familial insomnia, food poisoning, glanders, gonorrhea, Hepatitis A, Hepatitis B, Hepatitis C, Hepatitis D, Hepatitis E, human bocavirus infection, human eqingii ehrlichiosis, human papillomavirus, human parainfluenza virus, influenza, lyme disease, monkeypox, mumps, myiasis, pediculosis capitis, pneumonia, rabies, relapsing fever, rift valley fever, rotavirus infection, severe acute respiratory syndrome, shingles, smallpox, syphilis, tetanus, tinea cruris, tinea pedis, valley fever, viral pneumonia, West Nile fever, and yellow fever.

A device can include at least 1, 2, 3, 4, 5, 6, 7, 8, 9, 10, 15, 20, 25, 30, 35, 40, 45, 50, or more different samples. A device can include volumes of samples including at least about at least about 1 nanoliter (nL), 2 nL, 5 nL, 10 nL, 20 nL, 30 nL, 40 nL, 50 nL, 60 nL, 70 nL, 80 nL, 90 nL, 100 nL, 150 nL, 200 nL, 250 nL, 300 nL, 350 nL, 400 nL, 450 nL, 500 nL, 600 nL, 700 nL, 800 nL, 900 nL, 1 microliter (μL), 2 μL, 5 μL, 10 μL, 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, 1 milliliter (mL), 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, or 50 mL each.

### Pressure Modeling and Control

### Pressure Control

The pressure in a cavity of the device can be at least about 0.1 kilopascal (kPa), 0.2 kPa, 0.3 kPa, 0.4 kPa, 0.5 kPa, 0.6 kPa, 0.7 kPa, 0.8 kPa, 0.9 kPa, 1 kPa, 2 kPa, 3 kPa, 4 kPa, 5 kPa, 6 kPa, 7 kPa, 8 kPa, 9 kPa, 10 kPa, 15 kPa, 20 kPa, 25 kPa, 30 kPa, 35 kPa, 40 kPa, 45 kPa, 50 kPa, 55 kPa, 60 kPa, 65 kPa, 70 kPa, 75 kPa, 80 kPa, 85 kPa, 90 kPa, 95 kPa, 100 kPa, 110 kPa, 120 kPa, 130 kPa, 140 kPa, 150 kPa, 160 kPa, 170 kPa, 180 kPa, 190 kPa, 200 kPa, 210 kPa, 220 kPa, 230 kPa, 240 kPa, 250 kPa, 260 kPa, 270 kPa, 280 kPa, 290 kPa, 300 kPa, 310 kPa, 320 kPa, 330 kPa, 340 kPa, 350 kPa, 360 kPa, 370 kPa, 380 kPa, 390 kPa, 400 kPa, 410 kPa, 420 kPa, 430 kPa, 440 kPa, 450 kPa, 460 kPa, 470 kPa, 480 kPa, 490 kPa, or 500 kPa.

The pressure in a cavity of the device can be about 0.1 kilopascal (kPa), 0.2 kPa, 0.3 kPa,

0.4 kPa, 0.5 kPa, 0.6 kPa, 0.7 kPa, 0.8 kPa, 0.9 kPa,1 kPa, 2 kPa, 3 kPa, 4 kPa, 5 kPa, 6 kPa, 7 kPa, 8 kPa, 9 kPa, 10 kPa, 15 kPa, 20 kPa, 25 kPa, 30 kPa, 35 kPa, 40 kPa, 45 kPa, 50 kPa, 55 kPa, 60 kPa, 65 kPa, 70 kPa, 75 kPa, 80 kPa, 85 kPa, 90 kPa, 95 kPa, 100 kPa, 110 kPa, 120 kPa, 130 kPa, 140 kPa, 150 kPa, 160 kPa, 170 kPa, 180 kPa, 190 kPa, 200 kPa, 210 kPa, 220 kPa, 230 kPa, 240 kPa, 250 kPa, 260 kPa, 270 kPa, 280 kPa, 290 kPa, 300 kPa, 310 kPa, 320 kPa, 330 kPa, 340 kPa, 350 kPa, 360 kPa, 370 kPa, 380 kPa, 390 kPa, 400 kPa, 410 kPa, 420 kPa, 430 kPa, 440 kPa, 450 kPa, 460 kPa, 470 kPa, 480 kPa, 490 kPa, or 500 kPa.

The device can generate a number of distinct pressures including 1, 2, 3, 4, 5, 6, 7, 8, 9, 10, 11, 12, 13, 14, 15, 16, 17, 18, 19, 20, 25, or more distinct pressures.

A given pressure can be maintained for a specified time period. The specified time period can be at least about 1 millisecond, 10 milliseconds, 100 milliseconds, 1 second, 10 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 1 day, 5 days, 10 days, or 1 month. The specified time period can be at most about 1 millisecond, 10 milliseconds, 100 milliseconds, 1 second, 10 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 1 day, 5 days, 10 days, or 1 month. The specified time period can be about 1 millisecond, 10 milliseconds, 100 milliseconds, 1 second, 10 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 1 day, 5 days, 10 days, or 1 month.

In some examples, the pressure can be altered by changing the position of the pumping lid, without interrupting the experiment or process.

### Theoretical Modeling of Pumping Lid Operation

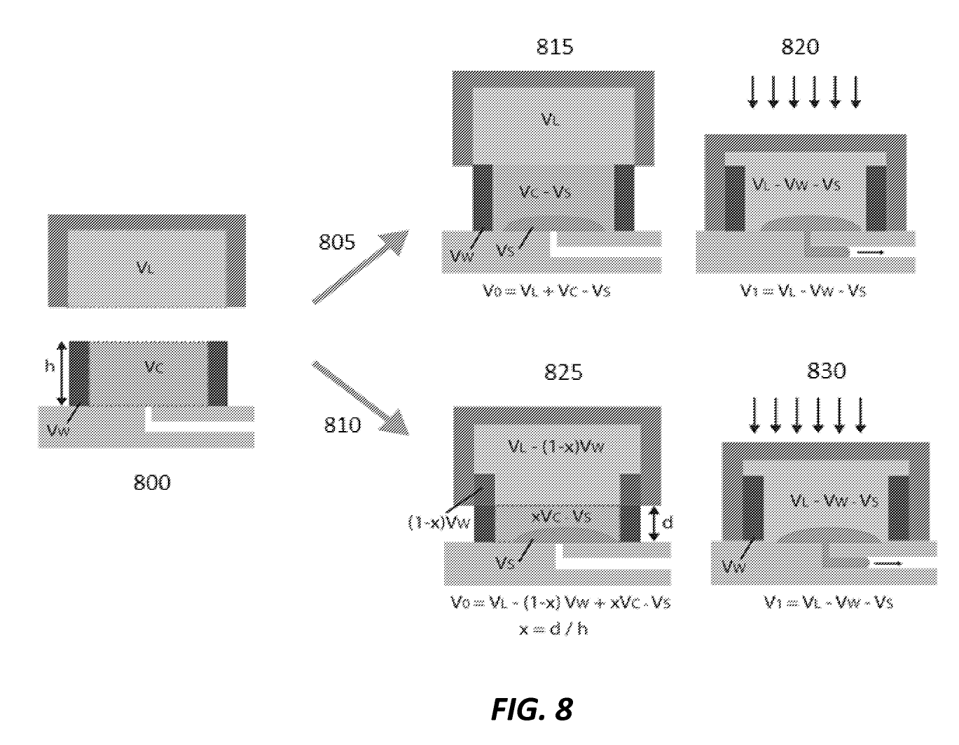

FIG. 8 provides an exemplary schematic representation of the parameters that can be used for the calculation of generation of positive pressure with a device disclosed herein. The device geometry 800 can be assembled in two ways. Option 805 is when the lid is placed around the vessel wall, as in schematic 815. The lid can be pushed and pumping can be started in step 820. The other option 810 is when the lid is pre-assembled on the vessel, as in 825. The lid can then be pushed and pumping started in step 830. It is apparent that different geometries, such as a cap that fits within the vessel walls would be require modification, e.g. to remove the contributions of the vessel wall.

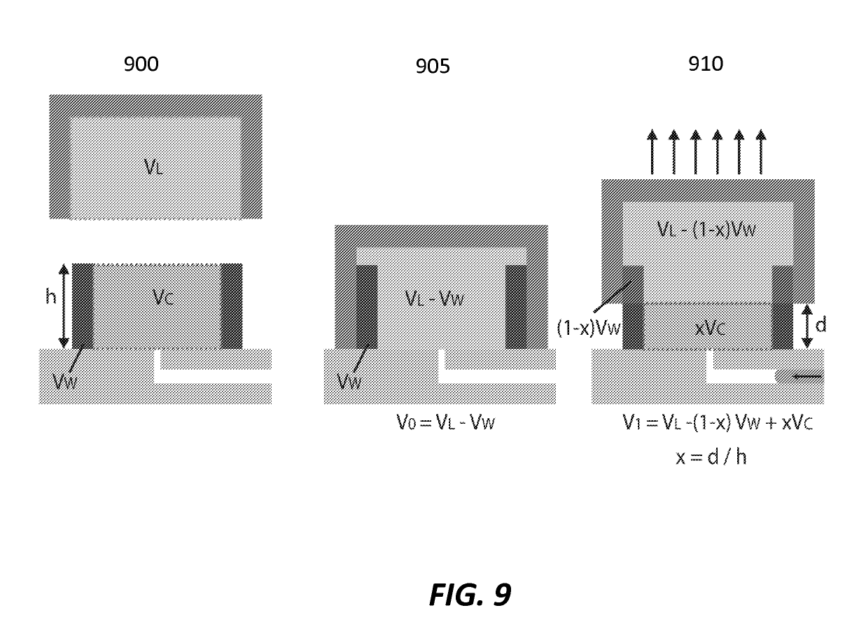

FIG. 9 provides an exemplary schematic representation of the parameters that can be used for the calculation of negative pressure with a device disclosed herein. The device geometry 900 shows the device before assembly, as in 905. The lid can be pulled and pumping started in step 910.

To model the pressure generated by the pumping lid method, in an implementation in which the lid fits around the exterior wall of the vessel, the initial pressure generated by the pumping lid and vessel, prior to pumping, may be analyzed. The Boyle law for isothermal gas compression may be used: P₀V₀=V₁V₁; assumptions of ideal gas behavior may be appropriate because the pressures are low (˜1 atm) and the temperatures are sufficiently high (˜300 K).

The positive pumping pressure may depend on four main parameters: the volume of the cavity in the pumping lid (V_{L}), the volume of the vessel walls (V_{W}), the volume of the empty space inside the vessel (V_{C}) and the volume of sample loaded in the vessel (V_{S}). When the lid is placed on the vessel and first creates the seal, the volume of air enclosed may be defined as V₀=V_{L}+V_{C}−V_{S}, and the initial pressure is P₀˜1 atm (FIG. 8, option 805). After the lid is pushed down, the air is compressed and the final volume may be given by V₁=V_{L}−V_{W}−V_{S}. Applying Boyle's law, the pressure at this point can be calculated as follows:

$${P}_{1} = \frac{{P}_{O} ( {V}_{L} + {V}_{C} - {V}_{S} )}{( {V}_{L} - {V}_{S} - {V}_{W} )} = {P}_{O} + \frac{{P}_{O} ( {V}_{W} + {V}_{C} )}{( {V}_{L} - {V}_{S} - {V}_{W} )} \qquad ( Eq . 3 )$$

A more generalized formula can be used for the case when the lid is already pre-placed on the vessel, at a distance d from the final position (FIG. 8, option 810). The pressure can be generated when the lid is moved down. The pressure calculated with the generalized model (Eq. 4) can be obtained assuming that the lid is pushed down by a distance d to its final position. The pressure can depend on the four volumes described above (V_{L}, V_{C} and V_{S}, V_{W}) and on the ratio x, between d and the total height of the vessel (h), defined as x=d/h. The initial volume can be given by V₀=V_{L}−(1−x)V_{W}+xV_{C}−V_{S} and the initial pressure is again assumed to be the atmospheric pressure, P₀˜1 atm. After the lid has been pushed down by a distance d, the final volume can be given by V₁=V_{L}−V_{W}−V_{S}. The pressure at this point can be calculated using the same relation, P₀V₀=P₁V₁, and can be defined as:

$${P}_{1} = \frac{{P}_{O} [ {V}_{L} + {xV}_{C} - ( 1 - x ) {V}_{W} - {V}_{S}^{0} ]}{{V}_{L} - {V}_{W} - {V}_{S}^{0}} = {P}_{O} + \frac{{P}_{O} x ( {V}_{W} + {V}_{C} )}{{V}_{L} - {V}_{W} - {V}_{S}^{0}} \qquad ( Eq . 4 )$$

V_{S}⁰ defines the initial sample volume. The changes in pressure due to pumping can then be analyzed. For the case described above, the pressure as a function of time can be expressed as:

$${P}_{1} ( t ) = \frac{{P}_{O} [ {V}_{L} + {xV}_{C} - ( 1 - x ) {V}_{W} - {V}_{S}^{0} ]}{{V}_{L} - {V}_{W} - {V}_{S} ( t )} \qquad ( Eq . 5 )$$

V_{S}(t) defines the volume of sample present in the vessel at time t. When the sample volume is substantially smaller than the difference between the cavity and pumping vessel volumes, V_{L}−V_{W}, the change in the only time-dependent term, V_{S}(t), becomes negligible and the pressure can be considered constant, and Eq. 5 becomes identical to Eq. 4. This assumption can be verified in all the experiments described, unless otherwise stated. Eq. 5 can be used to guide the design of pumping lids and vessels, to predict the variation in pressure due to pumping and tune it if needed.

When the sample volume is large enough to affect the pressure, the following set of equations can be used to describe the change in pressure. Given the hydraulic resistance (R_{H}) of the device, the time-resolved drop in positive pressure can be calculated as the sample is pumped out of the vessel:

$${P}_{1} ( t ) = \frac{{P}_{0} ( {V}_{L} - ( 1 - x ) {V}_{W} + {xV}_{C} - {V}_{S}^{0} )}{\sqrt{{( {V}_{L} - {V}_{W} )}^{2} + 2 ( \begin{matrix}\frac{{P}_{0} t}{{R}_{H}} ( {V}_{L} - ( 1 - x ) {V}_{W} + {xV}_{C} - {V}_{S}^{0} ) - \\ {V}_{S}^{0} ( {V}_{L} - {V}_{W} - \frac{{V}_{S}^{0}}{2} )\end{matrix} )}} \qquad ( Eq . 6 )$$

Eq. 6 can be valid for when P₁≧P₀ and while pumping is in progress. The values of R_{H} can be held constant in the experiments, as the channels with the solution being pumped can be pre-filled. If the channel is not pre-filled, the initial variation of R_{H} during filling might need to be accounted for. To calculate the time required to pump the whole sample volume, the following equation can be used:

$${t}^{*} = \frac{( {V}_{L} - {V}_{W} - \frac{{V}_{S}^{0}}{2} ) {V}_{S}^{0}}{\frac{{P}_{0}}{{R}_{H}} ( {V}_{L} - ( 1 - x ) {V}_{W} + {xV}_{C} - {V}_{S}^{0} )} \qquad ( Eq . 7 )$$

Eq. 7 relies on the same assumptions as Eq. 6.

For generation of negative gauge pressures, the pumping lid can be pre-placed onto the vessel, and pulled by a distance d. Assuming the vessel is empty prior to pumping, the initial volume can be given by V₀=V_{L}−V_{W}. The initial pressure can be assumed to be the atmospheric pressure, P₀˜1 atm. If the channel is not pre-filled with solution prior to pumping, the channel volume can be accounted for in V₀. After the lid has been pulled by a length d, the final volume of air can be given by V₁=V_{L}+xV_{C}−(1−x)V_{W}. Using previously defined parameters and the relation P₀V₀=P₁V₁, the pressure at this point can be defined as:

$${P}_{1} = \frac{{P}_{O} ( {V}_{L} - {V}_{W} )}{{V}_{L} + {xV}_{C} - ( 1 - x ) {V}_{W}} = {P}_{O} - \frac{{P}_{O} x ( {V}_{W} + {V}_{C} )}{{V}_{L} + {xV}_{C} - ( 1 - x ) {V}_{W}} \qquad ( Eq . 8 )$$

Similarly to the case of the positive pressure, once pumping commences, the time dependence of P₁ can be given by the expression:

$${P}_{1} ( t ) = \frac{{P}_{O} ( {V}_{L} - {V}_{W} )}{{V}_{L} + {xV}_{C} - ( 1 - x ) {V}_{W} - {V}_{S} ( t )} \qquad ( Eq . 9 )$$

V_{S}(t) represents the volume of sample pumped into the vessel at a given time t. When the sample volume is much smaller than V_{L}+xV_{C}−(1−x)V_{W}, the only time dependent term in Eq. 9, V_{S}(t), becomes negligible and the pressure can be considered constant. Whenever this assumption cannot be made, the time-resolved drop in pressure can be calculated as the sample is pumped into the vessel, given the hydraulic resistance (R_{H}) of the device:

$${P}_{1} ( t ) = \frac{{P}_{0} ( {V}_{L} - {V}_{W} )}{\sqrt{{( {V}_{L} - ( 1 - x ) {V}_{W} + {xV}_{C} )}^{2} - 2 \frac{{P}_{0} t}{{R}_{H}} ( {V}_{L} - {V}_{W} )}} \qquad ( Eq . 10 )$$

Eq. 10 can be valid when P₁≦P₀ and while pumping is in progress. To calculate the time required to pump a given sample volume, the following equation can be used:

$${t}^{*} = \frac{( {V}_{L} + {xV}_{C} - ( 1 - x ) {V}_{W} - \frac{{V}_{S}^{f}}{2} ) {V}_{S}^{f}}{( {V}_{L} - {V}_{W} )} \cdot \frac{{R}_{H}}{{P}_{0}} \qquad ( Eq . 11 )$$

V_{S}^{f} represents the total sample volume to be pumped into the vessel.

### Model for Volatile Material Evaporation Pressure Generation

To find the predicted pressure at volatile material equilibrium, the fugacities of perfluorohexane (e.g., FC-72) in both liquid (right hand side in Eq. 12) and gas (left hand side in Eq. 12) phases are set equal. The general expression for volatile material equilibrium is:

$${\hat{φ}}_{FC} {y}_{FC} p = {γ}_{FC} {x}_{FC} {φ}_{FC}^{sat} {P}_{FC}^{sat} exp [ \frac{{V}_{FC}^{L} ( P - {P}_{FC}^{sat} )}{RT} ] \qquad ( Eq . 12 )$$

Where: \hat{φ}_{FC}=fugacity coefficient of FC-72 in gas phase at T, P; y_{FC}=equilibrium mole fraction of FC-72 in the gas phase at T, P; P=equilibrium system pressure; γ_{FC}=FC-72 activity coefficient in liquid phase; x_{FC}=equilibrium mole fraction of FC-72 in the liquid phase at T, P; φ_{FC}^{sat}=fugacity coefficient for pure FC-72 at T, P_{FC}^{sat}, P_{FC}^{sat}=FC-72 saturation pressure at T, obtained from Antoine equation; V_{FC}^{L}=FC-72 liquid molar volume; R=ideal gas constant; and T=system temperature.

To simplify the calculation, the following assumptions can be made: Liquid phase is pure FC-72 (ignoring air dissolving in FC-72), x_{FC}=1; Liquid phase behaves ideally, γ_{FC}=1; Gas phase also behaves ideally, \hat{φ}_{FC}=1 and φ_{FC}^{sat}=1, and that Dalton's law applies: P=Σᵢpᵢ=pₐᵢᵣ+p_{FC}, where p_{FC}=y_{FC}P; T is constant.

After simplification, the equation becomes:

$${y}_{FC} P = {P}_{FC}^{sat} exp [ \frac{{V}_{FC}^{L} ( P - {P}_{FC}^{sat} )}{RT} ] \qquad ( Eq . 13 a )$$

Or, equivalently:

$${V}_{FC}^{L} ( P - {P}_{FC}^{sat} ) = RT ln ( \frac{P - {p}_{air}}{{P}_{FC}^{sat}} ) \qquad ( Eq . 13 b )$$

Because the Poynting factor (exponential term in Eq. 13a) is close to unity in experiments described in this document, the equilibrium system pressure P is almost equal to the initial pressure plus FC-72 saturation pressure. This equation was analysed numerically to calculate the predicted total pressure in the system (equal to P). If vapor pressure pumping is used in combination with the pumping lid approach, the final pressure P₁ should be used in place of pₐᵢᵣ.

The values of P_{FC}^{sat} were obtained with the Antoine equation:

$$ln ( {P}_{FC}^{sat} [ atm ] ) = 9.19734 - \frac{2488.59}{T [ ° C . ] + 213.42} \qquad ( Eq . 14 )$$

### Model for Temperature Dependence of Volatile Material Vapor Pressure

Vapor pressure of a volatile material, and therefore the performance of this pumping approach, is affected by temperature. To make accurate predictions of the pressure generated by this vapor pressure pump, the ideal gas law was substituted for pₐᵢᵣ (the initial pressure), which allowed us to take into account both the change in vapor pressure and gas expansion as the temperature is changed:

$${V}_{FC}^{L} ( P - {p}_{FC}^{sat} ) = RT ln ( \frac{P - \frac{{n}_{air} RT}{V}}{{P}_{FC}^{sat}} ) \qquad ( Eq . 15 )$$

Eq. 15 was used to calculate the predicted value of P at different temperatures. The total volume available to the gas phase in the device (V) can be calculated in CAD software. In these examples the initial number of moles of air in the gas compartment (nₐᵢᵣ) remained constant, and was therefore dictated by the temperature at which the compartment was initially sealed from atmosphere (21.5° C.). In these examples, the device was designed specifically to avoid any compression during the turning of the lid, to isolate the effects of volatile material equilibrium on pressure. For these volatile material equilibrium pumping experiments, the vapor pressure of the aqueous sample was neglected, because the vapor pressure of water is much lower than that of perfluorohexane (0.025 atm vs. 0.248 atm) at 21.5° C.

### Pressure and Flow Generation Using Volatile Material Vapor Pressure

The experimental behavior of pressure agreed with the theoretical predictions (see, e.g., FIG. 7C). The equilibrium pressure obtained experimentally approached the pressure predicted by the simplified volatile material equilibrium model (Eq. 13), and the system was used to pump 20 μL of water through a microfluidic device in ˜280 s (4.7 min). The volatile material equilibrium method can be used for pumping volumes in the milliliter range, for example 2 mL of water was pumped in more than 7 hours, showing less than 30% reduction in the input pressure using a lid with a 30 mL gas compartment (FIG. 7D). This reduction was caused by the fact that the volume previously occupied by sample became available to the gas phase to expand. As expected, larger lids took longer to equilibrate because more liquid needed to evaporate. However, the pressure remained stable when pumping was not in progress (FIG. 7D), so equilibration can be done prior to the pumping experiment. Alternatively, if the pressure does not need to be controlled precisely, the pumping can be started as soon as evaporation is initiated.

### Tuning of Volatile Material Vapor Pressure by Changing Composition or Temperature of the Volatile Material

Pumping pressures can be generated by materials with different vapor pressures. The equilibrium gauge pressure reached by the volatile material equilibrium system is related (but not necessarily equal) to the vapor pressure of the volatile material, according to Eq. 12. For a mixture of liquids, vapor pressure depends on the molar fraction of each component, amongst other factors. As an example, the equilibrium pressures for different mixtures of FC-40 (vapor pressure 0.003 atm at 21.5° C.) and FC-72 (vapor pressure 0.248 atm at 21.5° C.) were measured. Equilibrium volatile material pressure scaled linearly with the FC-72 molar fraction (R²=0.9999) and approached ˜0.003 atm for pure FC-40 (FIG. 7E), as expected. This method can be used with any combination of liquid, including volatile and non-volatile materials, to obtain the desired equilibrium pressure.

The pressure generated by this vapor pressure pump at different temperatures using FC-72 as the volatile material was investigated. Because vapor pressure is a function of temperature (Eq. 14 and Eq. 15), the equilibrium pressure of the example FC-72/air system increased with temperature, yielding values consistent with those predicted by the volatile material equilibrium model (FIG. 7F). The change in pressure with temperature far exceeded the one predicted for heating of an ideal gas in a closed volume. This presents an opportunity to incorporate simple microfabricated heaters to precisely control the pressures provided by this pump. As discussed herein, volatile material equilibrium pumping can potentially be used in combination with a pumping lid for gas compression or expansion. When generating positive pressure, the compression can be used to increase the range of pressures that can be achieved with the volatile material equilibrium approach. In the case of gas expansion, the use of volatile material equilibrium can set a lower limit to the pressure that can be obtained to the vapor pressure of the volatile material. The long-term stability of volatile materials in acrylic-based resins used for 3D-printing was not characterized, but preliminary experiments with the same liquids pre-packed in blister packs showed that it is possible to obtain similar pressures.

### EXAMPLES

### Example 1

### Generation of Predictable Positive and Negative Pressures

The principle of the pumping lid operation was tested. In these experiments, vessels were 3D-printed directly on a rigid support and not connected to a fluidic device. FIG. 1C shows experimental data of the pressures obtained from 40 combinations of vessels and pumping lids, plotted against the pressure value predicted by Eq. 4 and Eq. 8. The volume of the cavity was 15 mL, 20 mL, 35 mL, or 45 mL. The data indicates a linear fit.

A 5 psi differential pressure sensor (PXCPC-005DV, Omega Engineering), connected to a power supply (Portrans FS-02512-1M, 12V, 2.1 Amp power supply, Jameco Electronics) and to a data acquisition board (OMB-DAQ-2408, Omega Engineering) was used. A custom program was written in LabVIEW (National Instruments) to convert the signal collected by the sensor to gauge pressure. The sampling frequency was 2 Hz. Each condition varied in at least one model parameter (V_{L}: 14.7 mL-44.8 mL; V_{C}: 0-2.7 mL; V_{W}: 0.8 μL-3.6 μL; x: 0.25-0.75). The pumping lids used for these experiments included a nozzle that could be connected to the positive side of the pressure sensor using a short piece of Tygon tubing (1 cm long). Lid volumes were calculated using CAD software, accounting for the extra volume introduced by the nozzle, tubing, and the sensor. The other side of the sensor was exposed to the external environment, so all data collected were in terms of gauge pressure. The results were a close match to the predicted outcome, with an R² value of 0.9995 and a slope of 0.96. The pressures produced in this experiment spanned more than an order of magnitude. Furthermore, the model predicts that even higher pressure could be obtained by decreasing the volume of the empty parts (V_{L}, V_{C}) and/or by increasing the other volumes (V_{W} and V_{S}).

### Pressure Measurement Experiments

For the experiments described in FIG. 1A-FIG. 1C, four different pumping lids and five different vessels were used. All 20 combinations were tested for generation of positive and negative pressure (FIG. 1C). In this case, the vessels were printed directly on a rigid support and not connected to a microfluidic device. To ensure that the measured pressure was due to controlled expansion or compression of air, this rigid support had a venting hole that was closed with adhesive tape after the pumping lid was placed in its starting position. The experimental values of pressures measured with this approach were compared to the theoretical values calculated using the Eq. 4 and Eq. 8. For simplicity, no sample was placed in the cup during the reported experiments. The experimental conditions and predicted values of the generated pressure are reported in Table 2.

TABLE 2
Pressure measurements reported in FIG. 1C. The geometrical parameters (V_{C}, V_{R}, V_{E}, x) were used
to calculate the predicted gauge pressure value for both positive and negative pressures, according
to Eq. 4 and Eq. 8. These were compared to experimental values (mean ± S.D.) (N = 3).

| | | | | Positive Pressure | | | Negative pressure | | |
|---|---|---|---|---|---|---|---|---|---|
| | | | | Predicted | Experimental | | Predicted | Experimental | |
| | | | | Gauge | Gauge | Standard | Gauge | Gauge | Standard |
| V_{C} | V_{R} | V_{E} | | Pressure | Pressure | Deviation | Pressure | Pressure | Deviation |
| (μL) | (μL) | (μL) | x | (atm) | (atm) | (atm) | (atm) | (atm) | (atm) |
| 14730 | 3527 | 0 | 0.25 | 0.079 | 0.075 | 0.002 | −0.073 | −0.068 | 0.002 |
| 19746 | 3527 | 0 | 0.25 | 0.054 | 0.053 | 0.001 | −0.052 | −0.050 | 0.001 |
| 34795 | 3527 | 0 | 0.25 | 0.028 | 0.028 | 0.001 | −0.027 | −0.027 | 0.000 |
| 44828 | 3527 | 0 | 0.25 | 0.021 | 0.021 | 0.001 | −0.021 | −0.020 | 0.000 |
| 14730 | 3527 | 0 | 0.5 | 0.157 | 0.152 | 0.002 | −0.136 | −0.126 | 0.003 |
| 19746 | 3527 | 0 | 0.5 | 0.109 | 0.105 | 0.001 | −0.098 | −0.089 | 0.000 |
| 34795 | 3527 | 0 | 0.5 | 0.056 | 0.055 | 0.001 | −0.053 | −0.050 | 0.001 |
| 44828 | 3527 | 0 | 0.5 | 0.043 | 0.043 | 0.002 | −0.041 | −0.039 | 0.000 |
| 14730 | 3527 | 0 | 0.75 | 0.236 | 0.232 | 0.002 | −0.191 | −0.175 | 0.000 |
| 19746 | 3527 | 0 | 0.75 | 0.163 | 0.161 | 0.001 | −0.140 | −0.132 | 0.001 |
| 34795 | 3527 | 0 | 0.75 | 0.085 | 0.084 | 0.000 | −0.078 | −0.072 | 0.000 |
| 44828 | 3527 | 0 | 0.75 | 0.064 | 0.064 | 0.001 | −0.060 | −0.056 | 0.000 |
| 14730 | 3056 | 471 | 0.5 | 0.151 | 0.148 | 0.001 | −0.131 | −0.121 | 0.001 |
| 19746 | 3056 | 471 | 0.5 | 0.106 | 0.104 | 0.001 | −0.096 | −0.090 | 0.001 |
| 34795 | 3056 | 471 | 0.5 | 0.056 | 0.056 | 0.001 | −0.053 | −0.049 | 0.000 |
| 44828 | 3056 | 471 | 0.5 | 0.042 | 0.043 | 0.000 | −0.041 | −0.039 | 0.000 |
| 14730 | 813 | 2714 | 0.5 | 0.126 | 0.124 | 0.001 | −0.112 | −0.109 | 0.003 |
| 19746 | 813 | 2714 | 0.5 | 0.093 | 0.092 | 0.002 | −0.085 | −0.082 | 0.002 |
| 34795 | 813 | 2714 | 0.5 | 0.052 | 0.052 | 0.000 | −0.049 | −0.048 | 0.001 |
| 44828 | 813 | 2714 | 0.5 | 0.040 | 0.039 | 0.000 | −0.038 | −0.037 | 0.000 |

### Example 2

### Controlled Pressure Variation During an Experiment

It was demonstrated that it is possible to switch the pressure applied by the pumping lid without interrupting the flow or exposing the sample to the environment (to minimize contamination or evaporation). This capability can be desired when several flow rates need to be tested in one continuous experiment. Pressure can be changed by compressing or expanding air in the cavity. It was determined whether the level of compression or expansion, and therefore the pressure, can be controlled precisely by using the guiding structures (FIG. 2A-FIG. 2H). Guiding structures can be designed to produce one or more defined pressures (such as the ones described in FIG. 2A-FIG. 2H), or alternatively can enable a continuous range of pressure to be generated (for example by having a guiding structure that is designed as a continuous screw). As an illustration of this capability, for both positive- and negative-gauge pressures, lids were designed that had three potential positions, labeled (i) (ii) and (iii). Each position provides a defined, specific pressure, and the system can be switched between the positions by rotating the lid on its axis (FIG. 2D, FIG. 2H). For the examples shown in this section the lids for these experiments were 3D-printed with a nozzle for the pressure sensor and pressure data was collected with the same setup as described in previous sections. Measuring the pressure during the experiment is not required in general, but was used in this case for better characterization of the results. For both positive- and negative-pressure devices, the starting position, (i), was set corresponding to zero gauge pressure in this example FIG. 2A-FIG. 2H), but can be set at any pressure by changing the guiding structure geometry. The pressure can be reset by removing the lid from the vessel and placing it back at position (i). This adjustable design thus enables customized, “pre-programmed” pressure control during an experiment (e.g. to initiate or stop flow, and to change the flow rate) and allows the fully assembled device to be stored without applying pressure before use. While the devices demonstrated here are able to produce three specific pressures, more lid positions can be designed to enable finer tuning Also, guiding structures can be designed so that during a single experiment the pressure can be changed between positive, negative and zero.

### Example 3

### Generation of Flow Using the Pumping Lid Approach

The pumping lid approach can be used to pump fluids with predictable flow rate. Flow can be generated in any channel or compartment, including, for example, tubing, microfluidic channels, chambers, microfluidic chambers, and containers.

The prediction that for a given channel geometry, the pumping lid method would provide consistent flow rate that depends on viscosity, but not on surface energy or density of the fluid being pumped was tested. Eq. 3 was used to predict the pressure applied by the pumping lid, and Eq. 1 to predict hydraulic resistance R_{H} that depends on the viscosity and the dimensions of the channel.

$${R}_{H} = \frac{12 μL}{{h}^{3} w ( 1 - 0.63 ( \frac{h}{w} ) )} \qquad ( Eq . 1 )$$

L defines the channel length, h the channel height, and w the width of the channel. The volumetric flow rate can thus be predicted with Eq. 2:

$$Q = \frac{P}{{R}_{H}} = \frac{{Ph}^{3} w ( 1 - 0.63 ( \frac{h}{w} ) )}{12 μL} \qquad ( Eq . 2 )$$

To test these predictions, pumping of water through a microfluidic device using seven pumping lids was first characterized, each providing a different pressure (see, e.g., FIG. 3A). In this experiment the device consisted of glass-bonded PDMS layer, pumping vessel, PTFE tubing, and the pumping lid (see, e.g., FIG. 10). A 30.8 cm long, 58 μm high, 110 μm wide serpentine was molded into the PDMS layer, and was pre-filled with each solution prior to pumping experiment. The slope of the fitting curve is the inverse of the hydraulic resistance (R_{H}) for the experimental setup, as suggested by Eq. 2.

The experimental value for R_{H} obtained from the fit was 2.59*10¹⁴ Pa s/m³, which matched the theoretical value calculated for the microfluidic channel geometry: 2.58*10¹⁴ Pa s/m³. Thus, it is possible to predict the flow rate for a given pumping lid used with a given microfluidic device, and the design was robust enough to give reproducible results. The flow rates in this experiment were 1-5 μL/min. Higher flow rates can be produced by increasing the pressure generated by the pumping lid (as described in the previous sections), or by using a device with lower hydraulic resistance. For example, a device with a channel 150 μm tall×150 μm wide×20 mm long will have a hydraulic resistance almost 200 times less than the devices used for these experiments, so the flow rate generated with the same pumping lids would approach 1 mL/min.

### Microfluidic Device Fabrication (PDMS)

Devices used for flow experiments in this work were fabricated using rapid prototyping in PDMS from SU-8 photoresist molds. The devices were sealed by using a Plasma Prep II (SPI Supplies, West Chester, Pa.), and then baked overnight at 110° C. Vessels were connected to the PDMS devices by using adhesive transfer tapes (3M 468MP; Uline, Pleasant Prairie, Wis., USA), except in experiments involving fluorinated oils, where a silicone based adhesive (RTV 108 Translucent adhesive, Momentive performance materials, Columbus, Ohio, USA) was used.

### Flow Rate Experiments

The devices used for the flow rate experiment consisted of glass-bonded PDMS layer, cup, PTFE tubing, and the pumping lid (FIG. 10). A 30.8 cm long, 58 μm high, 110 μm wide serpentine channel was molded into the PDMS layer. The nominal hydraulic resistance for this device with pure water at 21.5° C. is 2.58*10¹⁴ Pa s/m³ (as calculated using Eq. 1). Prior to bonding to the glass slide, the PDMS layer was punctured (0.5 mm diameter) at the beginning and end of the serpentine channel. The 3D printed cup was attached to the other side of the PDMS layer with 3M 468MP transfer adhesive. A PTFE tubing (ID 356 μm) was connected to the device outlet, as shown in FIG. 10. The serpentine channel in the PDMS-glass device was pre-loaded with sample up to the point 1015. The cup was loaded with 50 μL of the same sample. The pumping lid was then pressed onto the cup, resulting in compression of air in the pumping lid cavity. The time it took the air-liquid interface to travel from point 1015 to point 1020 was recorded (point 1020 was 3.2 cm downstream from point 1015). Given the constant inner diameter of the PTFE tubing, the total volume pumped in that time was calculated to be 3.178 μL. This value was used to calculate the flow rate. The same device was used for all the flow rate experiments, with a DI water flush between different sample types. The density of the Tween-20 and Triton X100 solutions was assumed to equal that of pure water and viscosity was measured for all of the liquids using a M2-6 viscometer (Cannon Instrument Co., State College, Pa., USA). Results for nine sample types are shown in Table 3.

TABLE 3
Mean (±s.d.) pumping times and mean experimental flow rate,
Q, (±S.D.) of nine sample types (N = 3).

| | Water | | Tween-20 3.16e−6M | | Tween-20 3.16e−5M | |
|---|---|---|---|---|---|---|
| Pressure | Mean pumping | Q (±S.D.) | Mean pumping | Q (±S.D.) | Mean pumping | Q (±S.D.) |
| (atm) | time (±S.D.) (s) | (μL/min) | time (±S.D.) (s) | (μL/min) | time (±) (s) | (μL/min) |
| 0.199 | 37.7 (1.2) | 5.06 (0.17) | 37.0 (1.4) | 5.15 (0.20) | 37.3 (0.9) | 5.11 (0.13) |
| 0.174 | 43.3 (0.9) | 4.40 (0.10) | 43.0 (0.8) | 4.43 (0.08) | 43.0 (1.4) | 4.43 (0.15) |
| 0.145 | 51.3 (0.5) | 3.71 (0.03) | 50.7 (0.5) | 3.76 (0.04) | 51.3 (1.2) | 3.71 (0.09) |
| 0.124 | 62.0 (0.8) | 3.08 (0.04) | 62.0 (0.8) | 3.08 (0.04) | 62.0 (0.8) | 3.08 (0.04) |
| 0.094 | 83.3 (0.9) | 2.29 (0.03) | 81.3 (0.5) | 2.34 (0.01) | 80.0 (0.8) | 2.38 (0.02) |
| 0.069 | 113.3 (2.0) | 1.68 (0.03) | 112.7 (0.5) | 1.69 (0.01) | 109.7 (1.2) | 1.74 (0.02) |
| 0.049 | 173.7 (4.5) | 1.10 (0.03) | 168.3 (2.6) | 1.13 (0.02) | 163.0 (0.8) | 1.17 (0.01) |

| | Triton x100 1e−5M | | Triton x100 1.6 mM | | Glycerol 30 wt % | |
|---|---|---|---|---|---|---|
| | Average | | Average | | Average | |
| Pressure | Pumping time | Q (±S.D.) | Pumping time | Q (±S.D.) | Pumping time | Q (±S.D.) |
| (atm) | (±S.D.) (s) | (μL/min) | (±S.D.) (s) | (μL/min) | (±S.D.) (s) | (μL/min) |
| 0.199 | 38.0 (0.8) | 5.02 (0.11) | 38.0 (0.8) | 5.02 (0.11) | 82.0 (0.8) | 2.33 (0.02) |
| 0.174 | 44.7 (0.5) | 4.27 (0.05) | 43.3 (0.5) | 4.40 (0.05) | 95.7 (0.5) | 1.99 (0.01) |
| 0.145 | 52.0 (0.8) | 3.67 (0.06) | 52.0 (0.8) | 3.67 (0.06) | 111.0 (0.8) | 1.72 (0.01) |
| 0.124 | 62.0 (1.6) | 3.08 (0.08) | 61.7 (1.2) | 3.09 (0.06) | 136.3 (3.8) | 1.40 (0.04) |
| 0.094 | 81.3 (1.2) | 2.34 (0.04) | 78.3 (1.2) | 2.43 (0.04) | 180.7 (8.8) | 1.06 (0.05) |
| 0.069 | 115.7 (2.5) | 1.65 (0.04) | 107.3 (0.5) | 1.78 (0.01) | 248.0 (5.0) | 0.77 (0.02) |
| 0.049 | 169.3 (1.2) | 1.13 (0.01) | 155.7 (4.1) | 1.22 (0.03) | 379.0 (8.5) | 0.50 (0.01) |
| | Glycerol 50 wt % | | CsCl 3.66M | | CsCl 7.09M | |
| | Average | | Average | | Average | |
| Pressure | Pumping time | Q (±S.D.) | Pumping time | Q (±S.D.) | Pumping time | Q (±S.D.) |
| (atm) | (±S.D.) (s) | (μL/min) | (±S.D.) (s) | (μL/min) | (±S.D.) (s) | (μL/min) |
| 0.199 | 160.0 (2.1) | 1.19 (0.02) | 49.0 (1.4) | 3.89 (0.11) | 37.0 (1.4) | 5.15 (0.20) |
| 0.174 | 181.7 (2.0) | 1.05 (0.01) | 56.3 (1.7) | 3.38 (0.10) | 42.0 (1.4) | 4.54 (0.15) |
| 0.145 | 217.7 (3.1) | 0.88 (0.01) | 68.3 (1.9) | 2.79 (0.08) | 51.0 (1.4) | 3.74 (0.10) |
| 0.124 | 269.7 (6.3) | 0.71 (0.02) | 82.3 (0.5) | 2.32 (0.01) | 61.7 (0.9) | 3.09 (0.05) |
| 0.094 | 349.0 (5.7) | 0.55 (0.01) | 105.3 (0.5) | 1.81 (0.01) | 83.3 (1.9) | 2.29 (0.05) |
| 0.069 | 462.3 (5.2) | 0.41 (0.01) | 146.0 (2.2) | 1.31 (0.02) | 110.7 (1.9) | 1.72 (0.03) |
| 0.049 | 727.7 (21.5) | 0.26 (0.01) | 231.0 (4.2) | 0.83 (0.02) | 164.0 (2.1) | 1.16 (0.02) |

### Example 4

### Generation of Flow Rate Independent of Density and Surface Energy

Flow rate generated by the pumping lid method may be independent of solution density and surface energy. Nine aqueous solutions of different properties were used (Table 4) using seven different lids to measure the flow rate at different inlet pressures. Solutions of viscosity similar to water, but with different surface energies (30-72 mN/m) and different densities (1-1.9 g/mL), had flow rates comparable to those obtained for water. Viscosities of all nine solutions were measured experimentally to confirm this result. Note that the viscosity-adjusted flow rate values (Q·μ) were similar for all liquids (see, e.g., FIG. 3B).

TABLE 4
Properties of the liquids used in the flow rate experiments.

| | Surface Energy | Viscosity | |
|---|---|---|---|
| Aqueous solution | (mN/m) | (mPa * s) | Density (g/mL) |
| Water (DI) | 72.4 | 0.99 | 1.00 |
| Tween 20 3.16e-6M | 53 | 0.99 | 1.00 |
| Tween 20 3.16e-5M | 35 | 1.00 | 1.00 |
| Triton × 100 1e-5M | 57.5 | 1.00 | 1.00 |
| Triton × 100 1.6 mM | 30 | 1.00 | 1.00 |
| Glycerol 30 wt % | 71 | 2.10 | 1.07 |
| Glycerol 50 wt % | 69 | 3.98 | 1.11 |
| CsCl 3.66M | >75 | 0.93 | 1.47 |
| CsCl 7.09M | >75 | 1.24 | 1.90 |

### Example 5

### Generation of Flow for Solutions of Different Viscosities

Whether the pumping lid is appropriate to produce flow in solutions with viscosities higher than that of water was tested. In these experiments, solutions had viscosities between 1 mPa*s and 4 mPa*s (FIG. 3B). The flow rates for high viscosity solutions were lower than those obtained for pure water, because the value of the hydraulic resistance R_{H} described above is directly proportional to the viscosity of the liquid pumped (Eq. 1). Eq. 2 can be re-written as:

$$Q \cdot μ = \frac{{Ph}^{3} w ( 1 - 0.63 ( \frac{h}{w} ) )}{12 L} \qquad ( Eq . 16 )$$

Eq. 16 predicts that if the same lid-vessel combination is used on the same device, the product of the flow rate and the viscosity of the solution will be constant. Experimental results (FIG. 3B) corroborated this prediction, since the μ·Q values for all the solutions analyzed were comparable to those obtained for water (FIG. 3B). This means that the pressure generated by a pumping lid depended on the lid-vessel dimensions, and not on the nature of the solution to be pumped.

### Example 6

### Use of Multiple Lids on the Same Device to Achieve Complex Flow Control over Long Timescales

The idea that using separate vessels and lids at different inlets makes it possible to simultaneously pump more than one solution and to independently control the pressure imposed at each inlet (FIG. 4A) was tested. First, multiple lids were used to produce nanoliter droplets (FIG. 4B). Immiscible fluids can be difficult to handle under pressure-driven flow because the applied pressure should be higher than capillary pressure but not so high to generate an excessive capillary number that would cause droplet deformation. Also, when multiple inlets are controlled with different pressures, liquid could potentially flow from one vessel to another. To avoid this, devices with geometries that included a serpentine channel between the inlets and the junction can be used to produce the droplets. This serpentine channel had a fluidic resistance higher than that of the outlet channel, and ensured that liquids were not transferred from one vessel to the other during experiments. In one example, this approach was shown to generate nanoliter droplets (plugs) of water in fluorinated oil, using flow focusing and T-junction geometries (FIG. 4B), with volumes that ranged from 0.5 to 2.5 nL.

Parallel laminar flow profiles can also be produced (FIG. 4C). Stable flow patterns were obtained for more than 2.5 h, with a total pumped amount of 0.9 mL. The predicted decrease of flow rate in this system over a 2.5 h period was 45% of the original value (Eq. 6), which was consistent with experimental observations (see, e.g., FIG. 4C). Increased diffusion between the dyes was observed, due to the longer residence time in the channel. Because lids of the same size and loaded samples of the same volume and viscosity were used, over time a decrease in the absolute value of the flow rates, but not a decrease in their ratios, was observed. If the volumes of the lids, vessels, sample volumes and/or viscosities are different, the flow rates may drop at different rates (Eq. 6).

### Generating Droplets

Droplet generation experiments were performed using two geometries: flow focusing and T-junction. These devices were produced in PDMS by replica molding, bonded onto a flat layer of PDMS, and incubated at 110° C. for at least 24 hours to recover the hydrophobic properties of PDMS. Prior to each experiment the device was loaded with the inert, water-immiscible carrier fluid—a solution of perfluorodecaline (Acros Organics) and perfluorooctanol (Alfa Aesar), 9:1 volume ratio, as described previously.

### Flow Focusing

The geometry for flow focusing used in this work had two inlets (one for water and the other for the carrier fluid). Channels in the junction were 100 μm wide and 35 μm tall. The device included a serpentine channel (100 μm wide and 10.5 cm long) between each inlet and the junction, to increase fluidic resistance. A separate cup can be glued at each inlet. To generate droplets, a 100 μL sample of 0.5M FeSCN was placed in the cup at the water inlet and 100 μL of carrier fluid were placed in the other cup (FIG. 4A). The pumping lids were then placed on the vessels and pushed into final positions to generate flow. Pressures generated were 0.2 atm for the carrier fluid and 0.07 atm for the aqueous solution.

### T-Junction

The channel system for the T-junction used in this work included four inlets: three for water (in place of the single water inlet in FIG. 4A) and one for the carrier fluid. The three water channels included a serpentine, measured 50 μm tall and 10.5 cm long, and merged just before the T-junction (FIG. 4B, right). The channel used for the carrier fluid is 100 μm wide and 50 μm tall, and included a 10.5 cm long serpentine between the junction and the inlet. To generate droplets, 100 μL of one of the three solutions (0.5M FeSCM, pure water, and green food dye) were placed at the channel inlet in each of the three sample vessels, and 100 μL of the carrier fluid were placed in the fourth cup. The three sample inlets were controlled with the composite lid used in laminar flow experiments (composite lid 1, FIG. 5C), and the pressure applied to each of these inlets was 0.16 atm. The carrier fluid was controlled by a separate lid, producing a pressure of 0.2 atm.

### Laminar Flow Experiments

PDMS devices that were used for the laminar flow experiments had a constant channel height of 40 μm (FIG. 4C). Three inlets were included in each device, and each inlet was controlled by a separate cup (12 mm external diameter). The laminar flow patterns were monitored at the junction where the three channels (each 500 μm wide) merged into a single 1,500 μm wide channel. Between each of the three inlets and this junction, the device design included a serpentine channel (100 μm wide and 10.5 cm long) to increase hydraulic resistance. Experiments were performed by placing up to 300 μL of sample in each of the three vessels (0.5M FeSCM, pure water, and green food dye solution). Pressure could be produced by placing a different lid on each cup or by using a composite lid containing three apertures that align to each cup. For the experiments shown in FIG. 4C, three separate lids were used, each producing a pressure of 0.16 atm.

Five different composite lids were used to produce the five flow profiles shown in FIG. 5C. Based on the pressure applied to each inlet, the predicted flow profile can be calculated for each of the three streams (FIG. 5C; Table 5). The device geometry was such as the fluidic resistance of the channel between each of the three inlets and the junction (R) was significantly bigger than the fluidic resistance between the junction and the outlet. Under this condition, the flow rate in each branch can be calculated as

$${Q}_{i} \approx \frac{{P}_{i}}{R} .$$

TABLE 5
Calculated flow rates for the five lids used in the laminar flow experiments. The
pressure generated at each inlet was used to calculate the flow rate for each channel.
The hydraulic resistances R and r were 3.419 * 10¹⁴ Pa s/m³ and 8.745 * 10¹²
Pa s/m³, (values calculated according to Eq. 1). For each lid, the flow rate ratio
(Q₁/Qₜₒₜ) was calculated to plot the predicted flow profile in FIG. 5C.

| Lid | P₁ | P₂ | P₃ | Q₁ | Q₂ | Q₃ | Flow | Flow | Flow |
|---|---|---|---|---|---|---|---|---|---|
| Number | (atm) | (atm) | (atm) | (μL/min) | (μL/min) | (μL/min) | Ratio 1 | Ratio 2 | Ratio 3 |
| 1 | 0.160 | 0.160 | 0.160 | 2.61 | 2.61 | 2.61 | 0.33 | 0.33 | 0.33 |
| 2 | 0.068 | 0.347 | 0.068 | 1.00 | 5.88 | 1.00 | 0.13 | 0.75 | 0.13 |
| 3 | 0.347 | 0.036 | 0.347 | 5.77 | 0.32 | 5.77 | 0.49 | 0.03 | 0.49 |
| 4 | 0.347 | 0.068 | 0.068 | 5.88 | 1.00 | 1.00 | 0.75 | 0.13 | 0.13 |
| 5 | 0.068 | 0.068 | 0.347 | 1.00 | 1.00 | 5.88 | 0.13 | 0.13 | 0.75 |

### SlipChip Devices Fabrication and Experimental Procedure

The glass SlipChip device used for vacuum loading was produced by wet-etching of soda lime glass, using the protocol described in previous work and was provided by SlipChip Corp. The surface of the devices was treated with silane vapor to render it hydrophobic. Wells were etched at two different depths (40 μm and 100 μm) to obtain four different volumes: 1 nL, 5 nL, 25 nL, and 125 nL. The device also included a circular ring (100 μm deep and 4 mm wide), surrounding all the wells. Two through-holes were drilled in the top layer: the cup used for generating the vacuum was glued with 5-min epoxy (ITW Devcon, Danvers, Mass., USA) on the outlet hole, and a pierced PDMS piece (silicone rubber with adhesive back, 1.5 mm thick, McMaster Carr) was placed on the inlet hole to contain the sample during loading. Prior to device assembly, the pumping lid was placed on the cup, and the etched rings surrounding the wells were filled with high vacuum grease (Dow Corning) to ensure complete sealing of the active region of the device. Device assembly was performed in silicone oil (5 cSt, Sigma Aldrich). A 50 μL drop of 0.5 M FeSCN aqueous solution was then placed at the device inlet, and the pumping lid was pulled to produce ˜0.1 atm of negative gauge pressure and initiate the device loading. After loading by dead end filling was complete, a slipping step was performed to separate the sample into discrete droplets.

### Example 7

### Volatile Material Equilibrium Method for Pressure Generation

It was investigated the potential to harness the vapor pressure for pumping a non-volatile sample and whether the vapor pressure of a volatile material can aid the pumping process by isolating its effect from compression. It was hypothesized that (i) by taking advantage of volatile material equilibrium, large volumes of liquid could be pumped over extended periods of time at a relatively constant pressure, without the need to compress a large volume of a gas inside the device; (ii) a single lid design could be used to generate different pressures by using liquids of different vapor pressure; and (iii) a single combination of a lid design and a volatile material could be used to generate different pressures by tuning the temperature. In this approach, a volatile material can be stored in a sealed compartment. The compartment can be placed inside a vapor pressure pump, comprised of a lid and vessel (FIG. 7A). This vapor pressure pump can be pre-assembled. The sealed compartment containing the volatile material can be achieved using a combination of lid and vessel (FIG. 7A). The design of this lid and vessel differ from those described previously, as turning this lid connects or disconnects the compartments in the vessel, rather than compressing or expanding the gas enclosed in the cavity, as in a SlipChip device. In addition, the vessel used in these experiments was divided to contain the volatile material and one or more separate sample compartments. Confinement of volatile material can be achieved by the use of any sealed compartment approach, including, for example, sealed blister-packs. These compartments can be opened (for example by mechanical action) to initiate evaporation. In one example, a blister-pack containing a volatile material was placed in a lid/vessel assembly that incorporated protrusions inside the cavity. Turning the lid caused these protrusions to squeeze the blister-pack and released the volatile material, initiating evaporation.

In the case of the lid-vessel approach, when the lid is turned, the volatile material evaporates into the cavity (FIG. 7B). The cavity in the pumping lid is isolated from the atmosphere, so evaporation of the volatile material increases the pressure in the cavity. Once the volatile material reaches equilibrium with its vapor, the pressure will be higher than the atmospheric pressure, and its value can be calculated using the thermodynamic volatile material equilibrium model. Pumping can be initiated by opening a valve or removing a plug. During pumping, evaporation of additional liquid can provide additional pressure, although there is a drop in pressure, since the volume previously occupied by sample is now available to the gas phase, effectively causing expansion. This pressure drop can often be neglected, if the sample volume being pumped is much smaller than the pump gas compartment volume. Once the entire sample has been pumped through the device, the vapor in the lid can be connected to the atmosphere in which case the gauge pressure will drop. This method of vapor pressure pumping can be used independently or in conjunction with compression (e.g., pumping lid approaches described further herein).

### Vapor-Liquid Equilibrium Experiments

To harness vapor pressure for pumping, a different set of lids and vessels (vapor pressure pump) was designed, shown in FIG. 7A. The geometry and materials used are similar to the pumping lids described earlier, but in this case the cup is partitioned into separate compartments for liquid and gas. The gas compartment used in these experiments has an opening on the bottom that allows pumping through a PDMS device once the cup is bonded to it with 3M 468MP double-sided tape. The lid can be designed with a pressure-sensor nozzle. It also has a top opening for loading and pressure equilibration with the atmosphere. In the experiments described in this work, once the lid is put onto the cup, it can be turned to control the connection between different compartments and the atmosphere. The system was designed so that lid rotation did not induce compression in either compartment. The 5 psi differential pressure sensor (PXCPC-005DV, Omega Engineering) was used for real-time pressure monitoring.

In these experiments, the liquid compartment was filled completely with perfluorohexane (e.g., FC-72, Sigma Aldrich) (224 μL), sealed by the lid, and exposed to the gas compartment when the lid was twisted. To illustrate the broad range of sample volumes compatible with this method, results are shown for 20 μL and 2 mL. Samples were loaded into the gas compartment of the vapor pressure pump, at the inlet of the PDMS channel. In the case of the 2 mL experiment, a larger gas compartment was used, because the volume has to be large enough to accommodate the sample, and to reduce the pressure drop caused by pumping. The microfluidic channel was opened after the pressure equilibrated, although pumping can begin before equilibrium is reached. The equilibrium pressure for FC-72 at room temperature (21.5° C.), calculated using Eq. 13b, is 1.252 atm (corresponding to 0.252 atm gauge pressure).

To show that this approach can be used with a variety of pressures, and to illustrate one convenient way of tuning the equilibrium pressure, a modified device was utilized. In this case the gas compartment was 3D-printed without an outlet on the bottom. Mixtures of FC-72/FC-40 liquids of different molar ratios were used for these experiments, which were carried out by loading the compartmentalized cup, sealing with the lid without compression, and twisting the lid to connect the liquid and gas compartments, N=3 (FIG. 7E, Table 6). Pressure equilibration was monitored with the 5 psi differential pressure sensor (PXCPC-005DV, Omega Engineering)

TABLE 6
Experimental values for equilibrium pressures obtained with
mixtures of FC-72 and FC-40 (N = 3).

| | Average | |
|---|---|---|
| Molar | Equilibrium | Standard |
| Fraction | Gauge Pressure | Deviation |
| of FC-72 | (atm) | (atm) |
| 1.0 | 0.240 | 0.003 |
| 0.8 | 0.190 | 0.002 |
| 0.6 | 0.143 | 0.0005 |
| 0.4 | 0.095 | 0.001 |
| 0.2 | 0.047 | 0.002 |

The dependence of equilibrium pressure on temperature was tested using the same device. After loading and sealing the device, the inner partition between the gas and liquid compartments was removed by twisting the lid. All loading and sealing steps were done at 21.5° C. Then the device was placed in an incubator with an adjustable temperature, which was monitored in real time using a thermocouple (e.g., 5TC-TT-K-36-36, Omega Engineering). Both pressure and temperature were recorded through the same LabVIEW script at 2 Hz. Once volatile material equilibrium was reached at one temperature, the incubator was re-adjusted to a new temperature, and volatile material equilibrium was allowed to re-establish itself (Table 7). Data were compared to the predicted pressure calculated using Eq. 15 (FIG. 7F, Table 8).

### Example 8

### Multivolume Digital Nucleic Acid Amplification

In this example, a multivolume digital nucleic acid amplification design was first assembled with lubricating fluid (in this instance, 20% mineral oil in 80% tetradecane). FIG. 15A shows a schematic of an integrated C-clamp pumping vessel 1505, which can be made of a soft material, aligned over the inlet 1510 of a microfluidic glass SlipChip device 1515. The pumping vessel was pressed to the SlipChip device by tightening a 3-D printed screw 1520 into the bottom arm of the C-clamp 1500, but other tightening mechanisms may be used (such as fasteners, latches, clips, and others). During step 1525, the sample can be added and the lid applied, so that the lid 1530 can compress the chamber and increase the pressure within, thereby pushing the sample 1535 into a channel. This pressure, combined with a soft deformable material which was printed at the base of the pumping vessel, generates a seal and prevents leaks between the clamp and the glass device. Furthermore, this clamp can provide a clamping force to hold together the SlipChip and reduce the gap between the SlipChip plates. A 60 μL sample of 10% orange food coloring dye was added to the well and the pumping lid was applied to facilitate device loading (see, e.g., FIG. 15B). The vessel 1570 is placed over the screw 1580 and held in place with C-clamp 1585. The lid 1575 can be used to push a sample into the device. FIG. 15C shows a schematic of a pipette tip 1540 that can be used to deliver sample 1550 to a vessel 1555 mounted on a device 1545. During step 1560, the lid 1565 is applied, and positive pressure is produced to push the sample into the device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for altering a pressure in a chamber, the device comprising:
a) a guiding structure comprising a first part and a second part, wherein said first part comprises at least one docking structure and said second part comprises a guide that engages with said at least one docking structure, wherein said guide comprises (i) two or more docking positions for said at least one docking structure, and (ii) a pathway connecting a first docking position of said two or more docking positions to a second docking position of said two or more docking positions;
b) a lid comprising one of said first part or said second part of said guiding structure; and
c) a vessel comprising an open cavity and one of said second part or said first part of said guiding structure, such that between said lid and said vessel, said device includes a first part and second part of said guiding structure,
wherein said lid is capable of forming an airtight seal with said open cavity of said vessel, thereby defining a chamber,
wherein motion of said lid relative to said vessel is guided by said pathway, and
wherein, when said docking structure is in said first docking position, said chamber has a first volume and, when said docking structure is in said second docking position, said chamber has a second volume that is different from said first volume, wherein a change in volume produces a pressure change in said chamber.

2. A device for altering a pressure in a chamber, the device comprising:
a lid,
a cover, and
a vessel comprising one or more compartments, a first compartment of said one or more compartments containing a volatile material,
wherein an airtight seal is formed between said lid and said vessel, thereby defining a chamber,
wherein when said cover is in a first position, said cover obstructs fluid communication between said one or more compartments and said chamber, and when said cover is in a second position, said compartments are in fluid communication with each other and with said chamber and said volatile material produces a vapor pressure in said chamber.

3-5. (canceled)

6. The device of claim 1, wherein said lid further comprises a filter with a removable seal.

7-9. (canceled)

10. The device of claim 2, wherein said lid comprises said cover.

11-12. (canceled)

13. The device of claim 2, further comprising a port that is in fluid communication with at least one of said one or more compartments.

14-16. (canceled)

17. The device of claim 2, further comprising:
an additional lid,
an additional cover, and
an additional vessel comprising one or more compartments, a first compartment of said one or more compartments containing an additional volatile material,
wherein an airtight seal is formed between said additional lid and said additional vessel, thereby defining an additional cavity,
wherein when said additional cover is in a first position, said additional cover obstructs fluid communication between said compartments and said additional cavity, and when said additional cover is in a second position, said compartments are in fluid communication with each other and with said additional cavity and said additional volatile material expands into said additional cavity and increases a pressure in said additional cavity.

18-21. (canceled)

22. The device of claim 1, wherein said first part of said guiding structure comprises at least two docking structures.

23. The device of claim 1, wherein said guide comprises three or more docking positions.

24. The device of claim 1, further comprising a channel in fluid communication with said vessel.

25-35. (canceled)

36. A method of altering a pressure in a chamber, the method comprising:
a) providing a device, comprising:
a guiding structure comprising a first part and a second part, wherein said first part comprises at least one docking structure and said second part comprises a guide that engages with said at least one docking structure, wherein said guide comprises (i) two or more docking positions for said at least one docking structure, and (ii) a pathway connecting a first docking position of said two or more docking positions to a second docking position of said two or more docking positions;
a lid comprising one of said first part or said second part of said guiding structure; and
a vessel comprising an open cavity and one of said second part or said first part of said guiding structure, such that between said lid and said vessel, said device includes a first part and second part,
wherein an airtight seal is formed between said lid and said vessel, thereby defining a chamber, and
wherein motion of said lid is guided by said pathway; and
b) moving said lid from said first docking position to said second docking position, wherein a first volume in said chamber when said at least one docking structure is in said first docking position is different from a second volume in said chamber during said moving, wherein a change in volume produces a pressure change in said chamber.

37. A method of altering a pressure in a chamber, the method comprising:
a) providing a device comprising:
a lid,
a cover, and
a vessel comprising one or more compartments, a first compartment of said one or more compartments containing a volatile material,
wherein an airtight seal is formed between said lid and said vessel, thereby defining a chamber; and
b) moving said lid from a first docking position to a second docking position, wherein when said at least one docking structure is in said first docking position said compartments are not in fluid communication with each other or with said chamber, and when said at least one docking structure is in said second docking position said compartments are in fluid communication with each other and with said chamber and said volatile material produces a vapor pressure in said chamber.

38-39. (canceled)

40. The method of claim 36, further comprising moving said lid from said second docking position to a third docking position.

41-58. (canceled)

59. The method of claim 36, wherein said vessel holds a sample.

60. The method of claim 59, wherein said sample has a volume of less than about 1 mL.

61. The method of claim 59, wherein said sample has a volume of less than about 100 μL.

62-63. (canceled)

64. The method of claim 36, wherein said moving comprises rotating.

65-67. (canceled)

68. The method of claim 37, wherein said vessel holds a sample.

69. The method of claim 68, wherein said sample has a volume of less than about 1 mL.

70. The method of claim 68, wherein said sample has a volume of less than about 100 μL.
